(19) United States
(12) Patent Application Publication
(10) Pub. No.: US 2017/0121379 A1
(43) Pub. Date: May 4, 2017

(54) USING B-CELL-TARGETING ANTIGEN IGG FUSION AS TOLEROGENIC PROTEIN THERAPY FOR TREATING ADVERSE IMMUNE RESPONSES

(71) Applicant: THE HENRY M. JACKSON FOUNDATION FOR THE ADVANCEMENT OF MILITARY MEDICINE, INC., Bethesda, MD (US)

(72) Inventors: Ai-Hong ZHANG, Baltimore, MD (US); David SCOTT, Bethesda, MD (US)

(73) Assignee: THE HENRY M. JACKSON FOUNDATION FOR THE ADVANCEMENT OF MILITARY MEDICINE, INC., Bethesda, MD (US)

(21) Appl. No.: 15/309,026

(22) PCT Filed: May 7, 2015
(86) PCT No.: PCT/US2015/029642

§ 371 Date: Nov. 4, 2016

Related U.S. Application Data
(60) Provisional application No. 61/990,456, filed on May 8, 2014.

(51) Int. Cl. C07K 14/47; A61K 39/00; C07K 16/28

(52) U.S. Cl. CPC C07K 14/47; C07K 16/2887; A61K 39/0007; C07K 2317/622; C07K 2317/52; C07K 2319/33; C07K 2319/40; C07K 2319/30; A61K 2039/577; A61K 2039/6056

(57) ABSTRACT

The present invention generally relates to antigen-specific tolerogenic protein therapy and the use thereof for treating adverse immune responses, including those associated with autoimmune diseases such as multiple sclerosis (MS) and hemophilia. In particular, the invention involves the application of a B cell-targeting IgG fusion protein as the antigen-specific tolerogenic protein therapy, either alone or in combination with inhibitory antibodies. The fusion protein comprises a B-cell specific targeting module, the constant region of the human IgG4 heavy chain or a fragment thereof; and an antigen.

### RELATED APPLICATIONS

This application claims the benefit under 35 USC §119(e) to U.S. provisional application 61/990,456, filed May 8, 2014, the entire contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on May 1, 2015, is named 103783-0181_SL.txt and is 27,626 bytes in size.

### FIELD OF THE INVENTION

The present invention generally relates to antigen-specific tolerogenic protein therapy and the use thereof for treating adverse immune responses, including those associated with autoimmune diseases, such as multiple sclerosis (MS), and antibody responses to therapeutic proteins in hemophilia. In particular, the invention involves the application of a B cell-targeting IgG fusion protein as the antigen-specific tolerogenic protein therapy, either alone or in combination with, for example, inhibitory antibodies.

### BACKGROUND

B cells can play multiple roles in the pathology of multiple sclerosis. Current evidence suggests that B cells may contribute significantly to the pathogenesis of MS, but they also have regulatory function (see below). B cells may do this by producing CNS-specific pathogenic antibodies, as well as through pathogenic antigen presentation mediated by CNS antigen-specific B cells, or via antibody dependent cellular cytotoxicity (ADCC) locally in the CNS. The most direct evidence for the role of B cells in MS pathogenesis is that B-cell depletion therapy using rituximab was found to be beneficial in some relapsing-remitting multiple sclerosis patients during a phase II clinical trial.¹

However, the complete absence of B cells profoundly affects the normal immune response to infectious agents. More importantly, different subsets of B cells have been shown to have distinct immune functions. For example, antigen presentation by naïve resting B cells can be tolerogenic. Indeed, this mechanism may play an important role in maintaining peripheral tolerance in physiological conditions.² Furthermore, some B-cell subsets have recently been found to possess regulatory functions and depletion of these B cells may have unintended consequence.³ Therefore, complete B-cell depletion using reagents like rituximab might need to be reconsidered or modified for autoimmune diseases like multiple sclerosis.

Previously, it has been found that an IgG1 isotype anti-mouse CD20 mAb partially depletes B cells.^{4, 5} It depletes follicular (FO) B cells completely, but largely spares marginal zone (MZ) B cells, which can favor tolerance induction.⁵ Such partial B-cell depletion could potentially improve the therapeutic effect in multiple sclerosis. The other precedent for the invention is the previous successful preclinical application of “B-cell gene therapy approach for tolerance induction using engineered antigen-IgG fusion” in animal models for autoimmune disease, including CNS protein or peptide IgG fusion constructs for multiple sclerosis.^{6,7}

As stated above, although B cells can have a pathogenic role in multiple sclerosis (MS), complete B-cell depletion using anti-CD20 mAb drugs may not represent the best strategy: it lacks specificity and can cause severe side effects like infection.

Furthermore, self-tolerance to CNS antigens per se will not be restored simply by complete B-cell depletion.

MS traditionally has been considered to be a Th1 and Th17 T cell-mediated autoimmune disease, although CD8 and other effector cells may also be involved. Current available disease modifying drugs are mostly T cell-focused and can cause general immune suppression. However, the underlying immune pathogenesis of MS is likely to be due to a breakdown of peripheral tolerance to CNS myelin antigen(s), including myelin oligodendrocyte glycoprotein (MOG), myelin basic protein (MBP) or proteolipid protein (PLP). Thus, reliable methods to re-establish self-tolerance to the CNS antigens are needed to treat this disease.

Recently, the contribution of B cells in the pathogenesis of MS has been brought to attention, partly due to the beneficial effect of B-cell depletion therapy mediated by humanized anti-CD20 mAb, in some relapsing-remitting MS patients.¹ However, complete B-cell depletion using rituximab may not represent the best strategy for the treatment of MS, as discussed above. Complete B-cell depletion is not specific, and can cause severe side effects including increased susceptibility to infection. In addition, depletion of beneficial B cells (e.g., B cells with regulatory functions) may have unintended consequence. Ideally, a selective B-cell depletion agent, that depletes pathogenic B cells while sparing beneficial ones, would be a better choice.

A large body of evidence suggests that B cells are excellent tolerogenic antigen presenting cells, compared to antigen presentation by mature dendritic cells (DC). For example, Lassila et al.¹¹ first showed that resting B cells as APC were unable to activate resting T cells in vivo. Similarly, Eynon and Parker¹² demonstrated that resting B cells used as APCs led to tolerance to rabbit IgG. Using the male specific H-Y antigen as the model antigen, Fuchs and Matzinger¹³ subsequently demonstrated that both resting and activated B cells could be used as tolerogenic APCs to turn off a specific cytotoxic T lymphocyte (CTL) response, in naïve mice. In addition, specific subsets of B cells (i.e. MZ B cells) have been shown to be necessary for the systemic tolerance phenotype induced through an immune privileged site, such as the eye.¹⁴

One approach, used effectively by the Scott group during the last decade, utilizes transduced B cells for antigen-specific tolerance induction (FIG. 1). However, there is valid safety concern over retroviral vector mediated gene transfer in patients with autoimmune disease like MS.

MS has long been considered as primarily a CD4+ Th1 and Th17-mediated CNS autoimmune disease, although other lymphoid subsets have been implicated. Thus, current therapies often involve immune suppressive drugs that are focused on modifying T cell activity. Only recently has the important contribution of B cells in the pathogenesis of MS been brought to attention, partly due to the beneficial effect of B-cell depletion therapy using rituximab in relapsing-remitting MS patients in a phase II double blind clinical trial.¹ However, current therapeutic strategies generally lack methods to re-establish self-tolerance to the disease-causing CNS antigen(s).

Hemophilia is the second most common congenital bleeding disorder and is characterized by frequent bleeds at joint levels resulting in cartilage fibrosis, loss of joint space, and debilitation. Hemophilia affects the knees, ankles, hips, shoulders, elbows and bleeding into closed spaces can be fatal. Current treatment methods consist of infusions of either recombinant or plasma-derived clotting factor concentrates, usually in response to bleeds. Greater than 25% of hemophilia A patients make antibodies against therapeutic FVIII that inhibit clotting. Such conventional therapies require frequent injection/infusion of clotting factors over the patient's lifetime, and are associated with very high costs.

Accordingly, there is a need in the art to develop novel, effective therapies in treating adverse immune responses.

### SUMMARY

The present invention provides B cell-targeting IgG fusion proteins and methods of using the fusion proteins as antigen-specific tolerogenic protein therapy in the treatment of adverse immune responses.

One aspect of the present invention provides an isolated nucleic acid molecule comprising a nucleotide sequence encoding a fusion protein, wherein the fusion protein comprises an antigen, an IgG heavy chain constant region or a fragment thereof, and a B cell surface targeting molecule, as described herein. Vectors and host cells comprising the nucleic acid molecule are also provided.

The fusion protein may comprises an IgG heavy chain constant region that is a modified human IgG4 heavy chain constant region. In some embodiments the IgG4 heavy chain constant region lacks a hinge region. In other embodiments, the IgG4 heavy chain constant region lacks the CH1 region. In accordance with those embodiments, the antigen may be joined to the IgG4 heavy chain backbone by the hinge region of the IgG4 moiety.

In some embodiments, the fusion protein does not exhibit B cell depleting efficacy.

In some embodiments, the B cell surface targeting molecule of the fusion protein is an anti-CD20 single chain variable fragment, an anti-CD19 single chain variable fragment, an anti-CD22 single chain variable fragment, or an anti-CD23 single chain variable fragment. For example, the B cell surface targeting molecule may be a humanized anti-CD20, anti-CD19, anti-CD22, or anti-CD23 single chain variable fragment comprising an anti-CD20, anti-CD19, anti-CD22, or anti-CD23 variable heavy region linked to an anti-CD20, anti-CD19, anti-CD22, or anti-CD23 variable light region.

In some embodiments, the heavy and light chain regions are linked via a linker, such as a linker comprising the amino acid sequence (Gly-Gly-Gly-Gly-Ser)₃ (SEQ ID NO: 1). In some embodiments, the same type of linker is used to join the moieties of the fusion protein, e.g., the same type of linker is used to join the antigen to the IgG moiety and the IgG moiety to the B cell surface targeting molecule.

The antigen portion of the fusion protein may be any suitable target antigen depending on the condition to be treated, such as an antigen selected from the group consisting of myelin basic protein (MBP), myelin oligodendrocyte glycoprotein (MOG), proteolipid protein (PLP), Factor VIII C2 domain, Factor VIII A2 domain, and fragments thereof.

The components of the fusion protein may have different configurations with respect to the relative position of the antigen, the IgG moiety, and the B cell surface targeting molecule. In some embodiments, the fusion protein comprises, from the N-terminus to the C-terminus, the B cell surface targeting molecule, the IgG4 moiety, and the antigen (ScFv-IgG4H-Antigen). In other embodiments, the fusion protein comprises, from the N-terminus to the C-terminus, the B cell surface targeting molecule, the antigen, and the IgG4 moiety (ScFv-Antigen-IgG4H).

Another aspect of the present invention provides a method of inducing tolerogenicity to an endogenous protein in an individual by administering the fusion protein of the present invention or the isolated nucleic acid molecule of the present invention to said individual. In some embodiments, the method further comprises administering a B cell depletion agent. The B cell depletion agent may reduce the amount of all types of B cells. In some embodiments, the B cell depletion agent is rituximab.

In some embodiments the B cell depletion agent selectively reduces the amount of follicular B cells and does not reduce the amount of marginal zone B cells or reduces the amount of marginal zone B cells to a lesser extent that follicular B cells. In some embodiments, the B cell depletion agent is a human equivalent mouse IgG1 isotype anti-CD20 monoclonal antibody.

In some embodiments, tolerogenicity to an endogenous protein is induced wherein said endogenous protein is selected from the group consisting of MBP, MOG, PLP, Factor VIII C2 domain and Factor VIII A2 domain.

In some embodiments, the endogenous protein is MOG and the antigen of the administered fusion protein comprises amino acid residues 35-55 of MOG. In other embodiments, the endogenous protein is Factor VIII and the antigen of the administered fusion protein comprises amino acid residues 2191-2210 of Factor VIII.

In some embodiments, the method of the present invention may be employed in individuals that have been diagnosed with multiple sclerosis (using MOG, MBP, PLP, etc., as antigens), uveitis (using S-antigen, interphotoreceptor retinoid binding protein (IRBP), etc. as antigens), type 1 diabetes (using glutamic acid decarboxylase (GAD), islet-specific glucose-6-phosphatase catalytic subunit-related protein (IGRP), etc. as antigens), Myasthenia Gravis (using Acetylcholine receptor alpha (AChRα), etc. as antigens), hemophilia A or B (using Factor VIII or IX, respectively, as antigens) or a monogenic enzyme deficiency disease such as Pompe's (using acid alpha-glucosidase (GAA), etc. as antigens).

### BRIEF DESCRIPTION OF THE DRAWINGS

FIG. 1. Effect of gene therapy with MBP-IgG retrovirally transduced primed B cells on ongoing EAE. Protocol: Spleen and lymph node cells from MBP-immunized mice were expanded and transferred naïve syngeneic recipients, which were further boosted with MBP/CFA plus pertussis toxin to ensure increased disease incidence. Three days after transfer, mice received B cells transduced with MBP-Ig in a therapeutic protocol. Recipient mice were then monitored for EAE symptom daily and average disease score calculated. (Modified from Melo, et al. “Gene transfer of Ig-fusion proteins into B cells prevents and treats autoimmune diseases.” J. Immunol. 168: 4788-4795 (2002).)

FIG. 2A. Schematic of an exemplary B-cell-specific antigen IgG tolerogen. A single chain anti-CD20 mAb (VH-Linker-VL) is engineered on the N-terminus of the human IgG4 heavy chain constant region. The antigen is fused onto the C-terminus of the IgG4 CH3 domain. Optionally, the hinge between CH1 and CH2 domains is deleted through mutagenesis.

FIG. 2B. Schematic of an exemplary B-cell-specific antigen IgG tolerogen. An anti-CD20 single chain variable fragment (svCD20) is engineered to the N-terminus of the antigen-IgG4H fusion, where the antigen domain is connected with IgG4H backbone by the hinge region of the IgG4 lacking the CH1 region.

FIG. 3. Diagram showing a proposed mechanism of action and role of regulatory epitopes (Tregitopes) in tolerance induction. (From De Groot, et al., Blood 112: 3303-11 (2008).)

FIG. 4. Results of PCR cloning. Miniprep DNAs from the positive colonies were screened using restriction analysis. The expected size for the inserts was indicated. FIG. 4A: OVA₃₂₃₋₃₃₉ (antigen-specific control) and MOG₃₅₋₅₅ were successfully subcloned into pBSKS vector using SpeI and NotI restriction sites. FIG. 4B: eGFP and svCD20 were subcloned into pBSKS vector using SpeI/NotI and HindIII/EcoRI restriction sites, respectively. FIG. 4C: The hIgG4H was successfully PCR cloned from a human anti-FVIII IgG4 antibody producing line (2C11), and subcloned into the pBSKS vector using ECoRI and SpeI restriction sites. FIG. 4D: Restriction analysis of the subcloning vectors containing the full length inserts. Miniprep DNAs were prepared from the colonies for putative pBSKS-svCD20-IgG4H-X vectors. The DNAs were then digested with both Hind III and Not I restriction enzymes, followed by 2.0% agarose gel electrophoresis. Lane 1: 100 bp DNA ladder; lane 2: pBSKS-svCD20-IgG4H-MOG₃₅₋₅₅; Lane 3-5: pBSKS-svCD20-IgG4H-OVA₃₂₃₋₃₃₉; lane 6 & 7: pBSKS-svCD20-IgG4H-GFP. The size of the linearized empty pBSKS vector is about 3.0 kb. The expected size for the full length inserts, as indicated in the picture, are 1828, 1816, and 2485 bp, respectively.

FIG. 5: Schematic illustration for the BAIT expression cassettes. To achieve B-cell specific delivery of antigen-IgG for tolerogenic antigen presentation, single chain variable fragment (ScFv) against specific surface marker of B cells was engineered on the N-terminal of the fusions. The mBAIT fusion is based on anti-murine antibody sequence. To facilitate cloning and protein expression, a commercial available vector, pFuse-hIgG₄-Fc2 (Invivogen), was utilized. The ScFv-Antigen fragment was cloned into the pFuse-hIgG₄-Fc2 vector between the IL-2 signal sequence and the human IgG₄ hinge using the EcoR1 and EcoRV restriction sites as shown. Example of antigens used includes peptides FVIII₂₁₉₁₋₂₂₁₀ and MOG₃₅₋₅₅, as well as FVIII C2 and A2 domains. BAITs containing OVA₃₂₃₋₃₃₉ or OVA were used as the antigen specificity control. The G4S linker in this figure is set forth in SEQ ID NO: 33.

FIG. 6A: Restriction analysis of the pUC57-svCD19-MOG₃₅₋₅₅ vector by EcoRI and BamHI. The expected size of the insert is ˜817 bp.

FIG. 6B: Screening of the colonies for pFuse-msvCD19-MOG₃₅₋₅₅ (pFuse-mBAIT-MOG₃₅₋₅₅) by restriction analysis using EcoRI and EcoRV. The expected size for the insert is ˜817 bp. Four of the five screened colonies were positive. The plasmid DNA from the 1^{st} colony is selected for further analysis and experiment.

FIG. 7A: Restriction analysis of the intermediate vectors pUC57-svCD20-FVIII₂₁₉₁₋₂₂₁₀ and pUC27-svCD2O-OVA₃₂₃₋₃₃₉. The EcoRI/BamHI fragments were ˜829 by (lane 2) and ˜820 bp (lane 3), respectively. The inserts were then gel purified and cloned into EcoRI/BgIII digested pFuse-hIgG4-Fc2 expression vectors.

FIG. 7B: Screening of pFuse-BAIT vectors by restriction analysis using EcoRI and NcoI. The expected size for the inserts are ˜744 bp. All colonies screened contained right sized inserts. The plasmid DNA from 1^{st} of each of the screened pFuse-BAIT vectors were selected for further transfection and protein expression analysis.

FIG. 7C: Western blot analysis of the BAIT-FVIII₂₁₉₁₋₂₂₁₀ and BAIT-OVA₃₂₃₋₃₃₉ expression. CHO cells were transfected with either pFuse-BAIT-FVIII₂₁₉₁₋₂₂₁₀ or BAIT-OVA₃₂₃₋₃₃₉ plasmid DNA. The supernatant were collected 48 hrs after transfection and protein expression was analyzed by Western blot in NuPage 4-12% Bis-Tris gel. Under reducing condition, only one major band was detected at size of ˜56 KD for both of the proteins. Majority of the BAIT fusion proteins were in the form of polymers, as revealed by Western blot with non-reducing condition. The blotting antibody used was monoclonal anti-human IgG (λ, chain specific) and HRP Rabbit anti-mouse IgG (H+L).

FIG. 8A: Cell growth curve of the stably transfected CHO cell lines.

FIG. 8B: Western blot analysis of the supernatant samples from the stably transfected CHO cells in NuPage 4-12% Bis-Tris gel under reducing condition.

FIG. 9: The binding of BAIT-FVIII₂₁₉₁₋₂₂₁₀ to a CD20+ human B cell line, Raji cells, is shown. Raji cells (5×10̂5) were incubated with 1 μg of purified BAIT_{hCD20}-FVIII₂₁₉₁₋₂₂₁₀ for 1 hour at 37° C. The cells were then stained with APC anti-human IgG, which recognizes the human IgG4 Fc region of BAIT. The cells were gated on live singlet.

FIG. 10: The effect of B cell presentation of BAIT on the proliferation response of specific CD4+ effector T cells is shown. FIG. 10A demonstrates that the FVIII₂₁₉₁₋₂₂₁₀ epitope of the BAIT fusion was appropriately processed and presented to specific T cells by activated human B cells. Human B cells from a HLA DR1/DR2 donor were purified using anti-CD19 magnetic beads (Miltenyi) and activated with 2 μg/ml CD40L plus 10 ng/m IL-4 for 3 days. The activated B cells were then co-cultured with proliferation dye eFluor 450 labeled 17195 T effectors at the ration of 5:1, in the absence or presence of 1 μg/ml of either BAIT-FVIII₂₁₉₁₋₂₂₁₀, BAIT_{hCD20}-OVA₃₂₃₋₃₃₉, or recombinant FVIII. Three days after, the proliferation status of 17195 T effectors were evaluated by flow based on the dilution of the eFluor 450 fluorescence. FIG. 10B shows that resting B cells pulsed with BAIT_{hCD20}-FVIII₂₁₉₁₋₂₂₁₀ or FVIII did not support the proliferation response of specific T cells. Purified human B cells from a HLA DR1/DR2 donor were directly co-cultured with the labeled 17195 T effectors at the ration of 5:1, in the absence or presence of 1 μg/ml of either BAIT_{hCD20}-FVIII₂₁₉₁₋₂₂₁₀, BAIT_{hCD20}-OVA₃₂₃₋₃₃₉, or recombinant FVIII. The proliferation response of 17195 T effectors was evaluated as above. FIG. 10C shows that human PBMC pulsed with BAIT_{hCD20}-FVIII₂₁₉₁₋₂₂₁₀ did not support the proliferation response of specific T cells. Human PBMC from a HLA DR1/DR2 donor were co-cultured with the labeled 17195 T effectors at the ratio of 20:1, in the absence or presence of 5 μg/ml of either BAIT_{hCD20}-FVIII₂₁₉₁₋₂₂₁₀ or BAIT_{hCD20}-OVA₃₂₃₋₃₃₉, or 1 μg/ml of recombinant FVIII. The proliferation response of 17195 T effectors was evaluated as above.

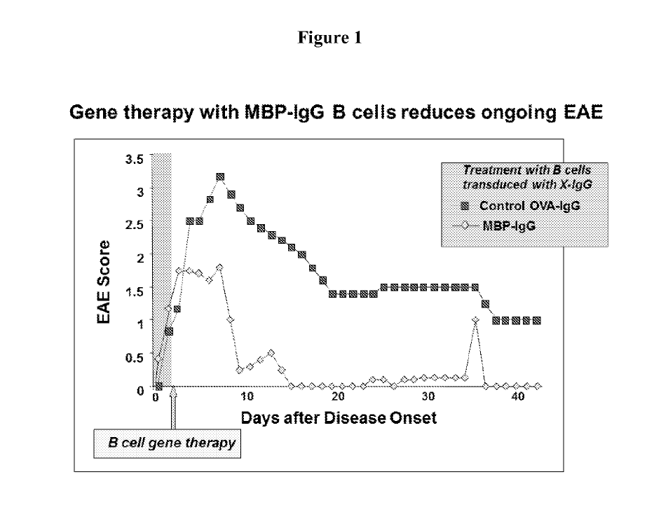

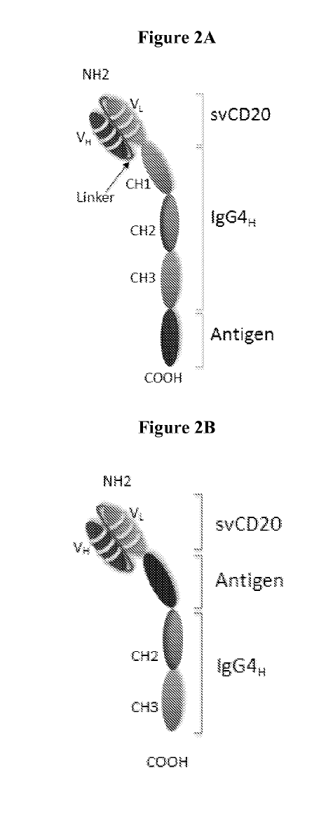

### DETAILED DESCRIPTION

The present invention relates to the application of a B cell-targeting IgG fusion protein as antigen-specific tolerogenic protein therapy in the treatment of adverse immune response, either alone or, for example, with inhibitory antibodies. The fusion protein is a B-cell-specific CNS antigen IgG fusion tolerogen (“BAIT”). Specifically, the invention provides a method of using B cell specific (targeting) antigen IgG fusion as tolerogenic protein therapy for treating adverse immune responses, such as in autoimmune diseases and hemophilia. The tolerance effect is achieved by exclusively using B cells as antigen presenting cells, and contributed by the inclusion of portions of IgG molecule in the fusion protein, which has immunomodulatory effects. This represents a novel therapeutic strategy relevant to the development of therapeutics that target B-cell lineages involved in, for example, multiple sclerosis pathology.

While aspects of the invention are discussed below in the context of MS, it is to be understood that the invention encompasses fusion proteins (and nucleic acid molecules encoding them, and methods using them) designed for use in the treatment of other diseases and conditions associated with an adverse immune response.

Three exemplary aspects of a tolerogenic BAIT protein may include: (1) the B-cell specific targeting module (such as an anti-CD20 single chain antibody (svCD20); (2) the constant region of the human IgG4 heavy chain (optionally with hinge deleted); and (3) the antigen, such as a CNS antigen, for example MOG₃₅₋₅₅.

Compared to existing fusion proteins for tolerogenic purpose, specific features of the present invention may include, but are not limited to, the following. First, the fusion protein on the present invention may exclusively target B cells. This is achieved, for example, by engineering anti-human CD20 single chain antibody (or other B cell-targeting moiety) into the fusion. Second, the IgG heavy chain portion of the fusion may be engineered so that the fusion does not have B-cell depleting efficacy like other anti-CD20 humanized antibodies do, and it does not fix complement. This will help preventing the capture of the antigen by other immunogenic antigen presenting cells. Third, application of this fusion can be in combination with other clinically used B-cell depleting agents, like rituximab. In such embodiments, traditional B cell depletion therapy will temporarily remove pathogenic B cells. The newly emerged naïve B cells can be ideal for mediating tolerogenic antigen presentation.

Aspects of the present invention include the use of isologous (self) immunoglobulins as carriers based on the tolerogenicity of IgG; an engineered target protein, e.g., autoantigens or FVIII domains, at the N-terminus of an IgG heavy chain scaffold; and transduced resting or activated B cells to produce or present fusion protein and act as tolerogenic APC.

B Cell Surface-Targeting Molecule

The BAIT fusion protein includes a B cell surface-targeting molecule, which may be based, for example, on CD20, -CD19, or CD23 targeting. In some embodiments, a BAIT fusion protein comprises a B-cell specific mAb selected from the group consisting of anti-CD19, -CD20, -CD22 or -CD23. Thus, the BAIT fusion protein may include a B cell surface-targeting single chain antibody (e.g., svCD19, svCD20, svCD22, svCD23). Such a fusion protein will be processed by naïve resting B cells and/or marginal zone B cells for tolerogenic antigen presentation. As discussed in more detail below, the tolerogenic effect is further promoted by including the constant region of human IgG heavy chain in the BAIT fusion protein, which may enhance regulatory T cell induction.⁸ Selective targeting of the BAIT fusion protein to naïve resting and/or MZ B cells could be facilitated by temporarily depleting FO B cells in advance, such as by using an anti-CD20 B-cell depletion agent. With this new strategy, not only may the disease symptoms be alleviated, but also the peripheral tolerance to culprit CNS self-antigens may be restored.

In some embodiments, the B cell surface targeting molecule is an anti-CD20 single chain variable fragment, an anti-CD19 single chain variable fragment, an anti-CD22 single chain variable fragment, or an anti-CD23 single chain variable fragment. The B cell surface targeting molecule may be a humanized anti-CD20, anti-CD19, anti-CD22, or anti-CD23 single chain variable fragment comprising an anti-CD20, anti-CD19, anti-CD22, or anti-CD23 variable heavy region linked to an anti-CD20, anti-CD19, anti-CD22, or anti-CD23 variable light region.

Sequences of single chain antibodies useful in the fusion proteins described herein are exemplified in the Sequence Listing, which includes DNA sequences for anti-mouse SvCD19 (original sequence) (SEQ ID NO: 2) and anti-mouse SvCD19 (codon optimized for expression in CHO cells) (SEQ ID NO: 3) and the translation thereof (SEQ ID NO: 4); and DNA sequences for anti-human SvCD20 sequence (original sequence) (SEQ ID NO: 5) and anti-human SvCD20 (modified and codon optimized for expression in CHO cells) (SEQ ID NO: 6), and translation thereof (SEQ ID NO: 7). It is within the purview of one of ordinary skill in the art to codon-optimize these and other sequences of single chain antibodies for use as B cell surface targeting molecules in the fusion proteins.

In some embodiments, the heavy and light regions are linked via a linker, such as a linker comprising the amino acid sequence (Gly-Gly-Gly-Gly-Ser)₃ (SEQ ID NO: 1).

Exemplary features of the BAIT fusion protein may include B cell specificity (with anti-human CD20 single chain antibody insert, for example a 729 bp, 243 amino acid insert). The anti-CD20 single chain antibody can be engineered at the N- or C-terminus of the construct so that it can fold and function properly in recognizing the CD20 molecule on the surface of B cells. Alternatively, anti-CD19 can be used for B-cell targeting. Targeting can be tested, for example, in human CD20 transgenic mouse.

An anti-CD20 single chain antibody sequence was engineered onto an antigen IgG fusion. Targeting the B-cell surface molecule CD20 is feasible, as B cell depletion using humanized anti-CD20 mAb, like rituximab, has been an FDA approved therapy for non-hodgkin's lymphoma and for certain types of multiple sclerosis. Alternatively, CD19 may be used as a target, and the fusion protein may be used in combination with traditional anti-CD20 mAb mediated B-cell depletion therapy.

IgG Heavy Chain

The BAIT fusion protein includes an IgG heavy chain, such an IgG heavy chain constant region that is a modified human IgG4 heavy chain constant region. In some embodiments the IgG4 heavy chain constant region lacks a hinge region. In other embodiments, the IgG4 heavy chain constant region lacks the CH1 region. In accordance with those embodiments, the antigen may be joined to the IgG4 heavy chain backbone by the hinge region of the IgG4 moiety.

In some embodiments, the IgG Fc region is engineered in a way that the fusion does not have any B-cell depletion property as normal antiCD20 mAb does, and that it will not fix complement. These measures are to prevent the potential transfer of antigen from surface of B cells to other immunogenic antigen presenting cells.

The presence of the IgG component may increase the half-life of the fusion protein in vivo. IgG Fc may contain immune regulatory elements, which could facilitate induction of regulatory T cells. Studies comparing a fusion protein containing an IgG heavy chain to a fusion protein that does not contain an IgG heavy chain can be conducted to determine the beneficial effects of each form (i.e., whether it is beneficial to include the IgG component).

Although applicant does not wish to be bound by theory, the rationale for including IgG heavy chain in the BAIT protein is as follows: first, it has been widely used as a tolerogenic carrier. In the 1970's, Borel and colleagues^{16, 17} demonstrated in adult animals that hapten-carrier conjugates were highly tolerogenic when some serum proteins were used as carriers. Of all serum proteins tested, immunoglobulin G (IgG) was the most tolerogenic. Indeed, Zaghouani and colleagues have utilized this principle to modulate EAE.⁹ In addition, well-conserved promiscuous epitopes were recently identified among different domains of IgGs; these have been shown to contribute to the induction of antigen-specific regulatory T cells.⁸ The constant region domains of human IgG4 were chosen because this human IgG isotype does not fix complement. In addition, the hinge region in the constant region of IgG4 will be deleted since IgG1 and IgG3 antibodies that lack a hinge region are unable to bind FcγRI with high affinity, likely due to the decreased accessibility to CH2¹⁸. Thus, the BAIT fusion targets B cells, but does not have the B-cell depletion effects of depleting anti-CD20 monoclonals.

Antigen

The antigen portion of the fusion protein may be selected from any suitable target antigens depending on the condition to be treated, as discussed in more detail below. Examples include, for autoimmune diseases, autoantigens. For example, in the case of MS, the CNS antigen MOG, PLP, MBP protein or peptide antigen can be used. For hemophilia A, Factor VIII or its domain may be used. Table 1 below sets for exemplary diseases and antigens.

TABLE 1
Summary of Applications Using Transduced B Cells for Tolerance

| Disease Model | Target Antigens | Results |
|---|---|---|
| Uveitis | IRBP, S-Antigen | Prevent disease, abolish transfer of disease |
| EAE (for multiple | MBP, MOG, PLP | Prevent EAE disease, block or reduce relapse, |
| sclerosis) | | abolish transfer of disease; stem cell therapy |
| Type 1 Diabetes | IGRP, GAD | Delay onset and reduce incidence of diabetes; |
| | | block transfer of disease; CD25+ T cells |
| | | needed for maintenance |
| Myasthenia Gravis | Acetylcholine | Modulate immune response to acetylcholine |
| | receptor alpha | receptor |
| Hemophilia A | Factor VIII C2 and | Prevent inhibitor formation in naive and |
| | A2 domains | primed recipients; CD25+ T cells needed for |
| | | induction |
| Hemophilia B | Factor IX | Prevent inhibitor formation in naive and |
| | | primed recipients²⁶ |
| Gene therapy | Target gene | Induce tolerance for long-term expression |

The sequences of antigen portions useful in the fusion proteins described herein are exemplified in the sequence listing, which includes DNA sequence for FVIII2191-2210 (codon optimized for expression in CHO cells) (SEQ ID NO: 8) and translation thereof (SEQ ID NO: 9); DNA sequence for OVA323-339 (codon optimized for expression in CHO cells) (SEQ ID NO: 10) and translation thereof (SEQ ID NO: 11), and DNA sequence for MOG35-55 (codon optimized for expression in CHO cells) (SEQ ID NO: 12) and translation thereof (SEQ ID NO: 13); DNA sequence for human FVIII A2 domain (codon optimized for expression in CHO cells) (SEQ ID NO: 14), and translation thereof (SEQ ID NO: 15); DNA sequence for human FVIII C2 domain (codon optimized for expression in CHO cells) (SEQ ID NO: 16), and translation thereof (SEQ ID NO: 17); and DNA sequence for chicken OVA (codon optimized for expression in CHO cells) (SEQ ID NO: 18), and translation thereof (SEQ ID NO: 19). It is within the purview of one of ordinary skill in the art to codon-optimize these and other sequences of single chain antibodies for use as B cell surface targeting molecules in the fusion proteins.

BAIT Fusion

The components of the fusion protein may have different configurations with respect to the relative position of the antigen, the IgG moiety, and the B cell surface targeting molecule. In some embodiments, the fusion protein comprises, from the N-terminus to the C-terminus, the B cell surface targeting molecule, the IgG4 moiety, and the antigen (ScFv-IgG4H-Antigen). In other embodiments, the fusion protein comprises, from the N-terminus to the C-terminus, the B cell surface targeting molecule, the antigen, and the IgG4 moiety (ScFv-Antigen-IgG4H).

In some embodiments, the same type of linker is used to join the moieties of the fusion protein, e.g., the same type of linker is used to join the antigen to the IgG moiety and the IgG moiety to the B cell surface targeting molecule. It is within the purview of one skilled person to select a suitable linker which can permit or promote the proper folding of the fusion protein and enable efficient secretion. In some embodiments, one or more of the linker(s) comprise the amino acid sequence (Gly-Gly-Gly-Gly-Ser)₃ (SEQ ID NO: 1).

The invention includes an IgG fusion protein with a single chain anti-CD20 (SvCD20) (or other B cell-targeting molecule) containing the target antigen engineered either at N- or C-terminus. The approach of the present invention to employ a single chain antibody to construct an IgG heterodimer in which one half is specific for a B cell surface protein and the other is a fusion with the targeted epitope (antigen) (which may be, for example, at the N-terminus of the fusion or between the B cell-targeting molecule and IgG moiety) allows for the expression of a targeted epitope, which is a much simpler and less expensive application than the use of retroviruses. Thus, it is more commercially appealing than gene therapy per se.

The present invention additionally encompasses methods to generate and characterize BAIT fusion proteins and to determine the tolerogenic effect of the BAIT fusion protein used in combination with/without the aforementioned selective B-cell depletion agent in a mouse model for MS.

In some aspects, three BAIT proteins varying in the antigen moiety are generated, such as, for example: BAIT-MOG₃₅₋₅₅, BAIT-OVA₃₂₃₋₃₃₉ (antigen-specificity control), and BAIT-GFP (for in vitro characterization). A MOG-IgG (i.e., MOG₃₅₋₅₅ on a normal, non-specific IgG backbone) is used as a control. Construction of the vector for BAIT molecules will be divided into three steps. First, each of the three modules will be cloned into a pBSSK vector, used previously.¹⁰ Then, pBSSK-svCD20-hIgG4-Ag will be generated based on the pBSSK-svCD20 vector. Finally, the entire svCD20-hIgG4-Ag fragment will be ligated into a mammalian expression vector, pSec-Tag2A. The resulting vector will be pSec-svCD20-hIgG4-Ag-Tag2. Expression and purification of the BAIT protein will be according to established procedures. The purified BAIT proteins will be extensively characterized as described below.

Alternatively, various expression cassettes differing in the B-cell targeting moiety are cloned into a commercially available hIgG4 fusion protein expression vector, such as pFuse-hIgG4-Fc2 (Invivogen). Subsequently, the BAIT protein is expressed and purified according to established protocols.

Table 2 below shows a list of exemplary BAIT vectors. The mBAIT vectors are based on murine antibody sequences.

| BAIT vectors | mBAIT vectors |
|---|---|
| pFuse-BAIT-MOG₃₅₋₅₅ | pFuse-mBAIT-MOG₃₅₋₅₅ |
| pFuse-BAIT-FVIII₂₁₉₁₋₂₂₁₀ | pFuse-mBAIT-FVIII₂₁₉₁₋₂₂₁₀ |
| pFuse-BAIT-OVA₃₂₃₋₋₃₃₉ | pFuse-mBAIT-A₂ |
| | pFuse-mBAIT-C₂ |
| | pFuse-mBAIT-OVA₃₂₃₋₃₃₉ |
| | pFuse-mBAIT-OVA |

Methods

In some embodiments, the BAIT fusion protein therapy is employed by itself as a stand-alone effective therapy. In some embodiments, a selective B-cell depletion approach is employed without BAIT fusion protein therapy. In some embodiments, the present invention involves combining a tolerogenic BAIT fusion protein therapy with partial B-cell depletion. In some embodiments, the therapies of the present invention are used to treat MS, or other autoimmune conditions, or other conditions discussed herein.

In one aspect of the present invention, the BAIT fusion protein, used either alone or in combination with, for example, a selective B-cell targeting agent, can re-establish immunologic self-tolerance and ameliorate the disease symptoms, such as for multiple sclerosis (MS).

Thus, in some aspects, the present invention employs two interrelated strategies: One is targeting pathogenic B cells for elimination using a selective B-cell depletion agent, such as IgG1anti-mouse CD20 mAb. For example, it has been found that an IgG1 isotype murine anti-mouse CD20 mAb partially depletes B cells.^{4, 5} Thus, the IgG1 isotype depletes follicular (FO) B cells completely, but largely spares marginal zone (MZ) B cells, which is believed to favor tolerogenic antigen presentation. The second one is targeting beneficial B cells for tolerogenic antigen presentation, facilitated by the novel BAIT fusion proteins described herein. As a tolerogenic regime, BAIT may complement the current rituximab mediated B-cell depletion therapy and/or other MS disease modifying drugs. As new selective B cell depleting reagents are developed, these can be integrated into the developmental protocol.

In some embodiments, a newly developed humanized anti-CD20 mAb which partially depletes B cells is more effective in controlling disease with fewer side effects than Rituxan. BAIT(s) will not compete with Rituxan, but will complement it by targeting newly emerged naïve resting B cells after Rituxan treatment to serve as tolerogenic antigen presenting cells and re-establishing self-tolerance to associated CNS antigens.

The BAIT fusion protein therapy described herein can be adapted as a platform for tolerogenic protein therapy for MS. It is known that CNS fusion IgGs per se (e.g., MOG in frame with a non-specific generic IgG: MOG-IgG) can be tolerogenic.⁹ A fusion protein that selectively targets uptake by B cells, such as the BAIT fusions described herein, may be more effective tolerogens. In addition, combining a selective B-cell depletion therapy with the tolerogenic BAIT fusion protein comprising a CNS antigen may be more effective at reducing clinical symptoms. This will be shown by the Experimental Autoimmune Encephalomyelitis (EAE) model using MOG-peptide induction of EAE in human CD20 transgenic mice (hCD20, C57Bl/6 background).

Thus, to modulate the fundamental autoimmune features of the MS, a novel B-cell-specific tolerogenic fusion protein, BAIT is generated and characterized. BAIT fusion proteins can be administered alone or during the recovery phase after a B-cell depleting round of therapy. In addition, complete B-cell depletion by rituximab does not necessarily represent the best strategy for targeting pathogenic B cells. Indeed, depleting beneficial antigen-specific B cells and regulatory B cells may have unintended consequences.⁵ This explains, in part, the side effects seen in patients treated with rituximab, such as infection or even Progressive Multifocal Leukoencephalopathy. Accordingly, a partial B-cell depleting agent, IgG1 isotype anti-mouse CD20 mAb that depletes FO B cells completely while largely sparing tolerogenic MZ B cells, is provided. Thus, some embodiments of the present invention combines two innovative B-cell targeting methodologies with distinct purposes. Partial B-cell depletion mediated by IgG1 anti-mouse CD20 will temporarily eliminate FO B cells, which contain pathogenic B cells; while the BAIT fusion protein will be targeted to newly generated naive resting B cells and the remaining tolerogenic MZ B cells for tolerogenic antigen presentation to CD4+ T cells. Thus, the invention includes humanized anti-CD20 mAb with selective B-cell ablation activity, such as BAIT fusion proteins. While it is possible that either BAIT or selective B-cell depletion can act as stand-alone therapies, combination therapies may offer advantages. Each of the possibilities will be examined in detail in the experiments below using a mouse model of MS.

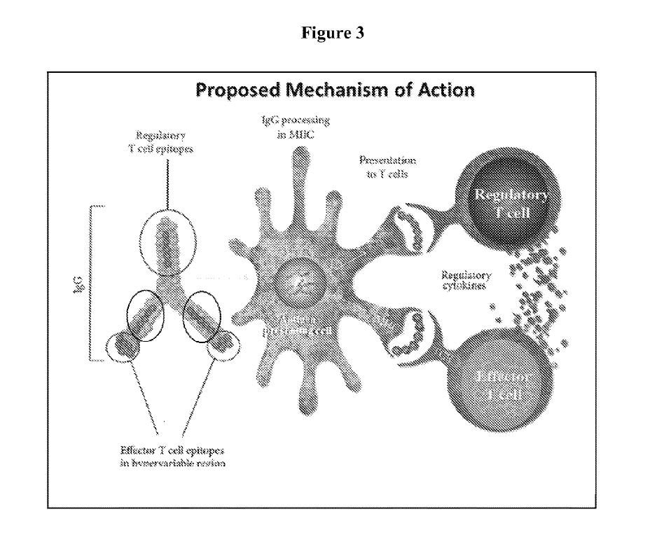

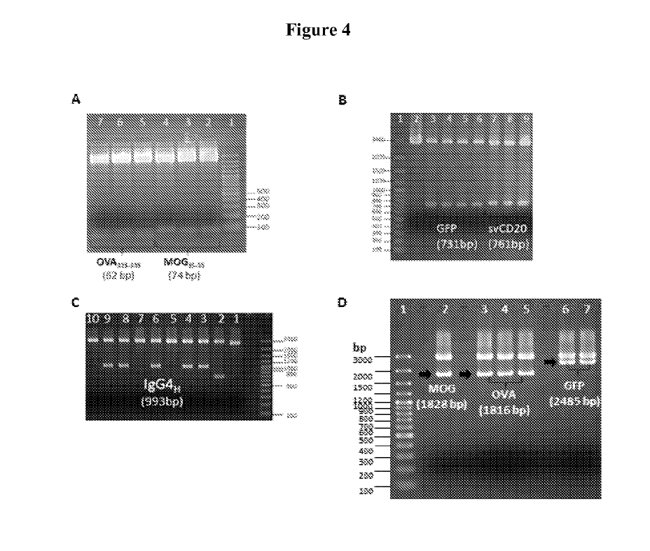

Taking the BAIT protein depicted in FIG. 3B as an example, three exemplary properties may be embodied in the BAIT protein: an anti-human CD20 single chain antibody (svCD20) to direct the fusion protein specific to B cells; a constant region of human IgG4 heavy chain to enhance the tolerance effect without activating effector functions via FcR or complement; and an antigen module, to present to targeted T cells and induce antigen-specific tolerance. The svCD20 was originally cloned from B9E9 hybridoma cells expressing murine IgG2a anti-CD20. The svCD20 is comprised of linked V_{H} and V_{L} chains from the anti-CD20 with a (Gly₄Ser₁)₃ linker (SEQ ID NO: 1) between them.¹⁵ The svCD20 sequence in the BAIT protein is the same as that encoded by a lentiviral vector system.

Additionally selective B-cell depletion, such as one that spares beneficial B cells, will have a greater efficacy in the treatment of MS compared to that of complete depletion. It has been shown that an IgG1 isotype anti-mouse CD20 mAb only partially depletes peripheral B cells^{4, 5}. While the IgG2a isotype anti-mouse CD20 mAb completely depletes both FO and MZ B cells, the IgG1 anti-mouse CD20 only depletes FO B cells, but largely spares MZ B cells, and favors tolerance.⁵ In fact, the IgG2a antibody did not facilitate tolerance. The reason MZ B cells are spared by IgG1 isotype anti-mouse CD20 is presumably due to the inability of this mouse IgG subclass to fix complement, since C3 activation is a requirement for depletion of MZ B cells using anti-CD20.¹⁹

Thus, therapy using a BAIT MOG₃₅₋₅₅ tolerogenic protein will target naïve B cells to effectively present CNS epitopes for tolerance. Moreover, the combination therapy that spares MZ B cells after selective B-cell depletion will help re-establish the CD4+ T cell tolerance to the CNS antigen and ameliorate disease symptoms in a mouse model for MS.

### EXAMPLES

### Example 1

### Generation and Characterization of the BAIT Fusion Protein

Overall Strategy

One of the key steps in this proposed study is to optimize the design of the BAIT molecule and generate fusion proteins with desired characteristics. Three basic modules of the BAIT molecule will be: (1) the B-cell targeting module, (2) portions of IgG4 heavy chain constant region, and (3) CNS antigen module. For the B-cell targeting module, an anti-CD20 single chain antibody, cloned from an anti-CD20 mAb B9E9, will be engineered onto the N-terminus of the molecule. Alternatively, anti-CD19 single chain antibody sequence can be used.

As mentioned above, the BAIT fusion protein is not designed to target B cells for depletion. Instead, it is used as a vehicle to direct the tolerogen into B cells for tolerogenic antigen presentation. Therefore, complement activation, opsonization and ADCC functions mediated by IgG Fc region are not desirable in the design of BAIT molecule. For this reason, the heavy chain constant region of human IgG4 will be used, as IgG4 does not fix complement. In addition, the hinge region of the IgG4 heavy chain will be mutated to disable the FcγR1 binding activity.

For the antigen module, the encephalitogenic MOG₃₅₋₅₅ sequence will be used. MOG₃₅₋₅₅ is quite conserved in mice and humans, so the tolerogenicity of the BAIT fusion protein can be tested in the MOG₃₅₋₅₅/CFA induced mouse EAE model, and the product will be utilizable in future clinical trials. Further, convenient cloning sites will be engineered to flank the antigen module, so that other human CNS antigens can easily replace the MOG₃₅₋₅₅ encoding DNA, after the proof of principle experiments.

Each component will be PCR cloned and inserted into a pBSSK vector individually. High fidelity Taq DNA polymerase (Invitrogen) will be used for all PCR reactions. First, the pBSSK-svCD20-hIgG4-MOG₃₅₋₅₅ will be constructed using the indicated restriction sites. Then, the mammalian expression vector pSec-Tag2-svCD20-hIgG4-MOG₃₅₋₅₅ will be constructed by cloning the entire fusion fragment from the pBSSK vector into a pSec-Tag2 vector.

Alternatively, various expression cassettes differing in the B-cell targeting moiety were cloned into a commercially available hIgG4 fusion protein expression vector, such as pFuse-hIgG4-Fc2 (Invivogen). Detailed cloning procedures and the subsequent characterization are as below.

Design and Construction of pBSSK-svCD20-hIgG4-CNS Antigen (BAIT) Molecule

pBSSK-svCD20

Oligonucleotide primers, CD20-forward 5′-agaggaagcttatggctcaggttca-3′ (SEQ ID NO: 20) and CD20-reverse 5′-agagcgaattccttcagctccagct-3′ (SEQ ID NO: 21), were designed. HindIII and an EcoRI sites, indicated in italics, were included in the forward and reverse primers, respectively. In addition, a single nucleotide “g” was added in the forward primer between the HindIII site and the svCD20 sequence to maintain frame. Using a pCG-H-αCD20 vector (kindly provided by Kneissl S and Buchholz C J) as the template, the primer pair will amplify a fragment encoding the svCD20 derived from a mouse anti-human CD20 mAb clone B9E9. The PCR fragment will be cloned into the pBSSK vector using the HindIII and EcoRI restriction sites.

pBSSK-hIgG4

RNA will be isolated from a human B-cell hybridoma which secrets IgG4 anti-factor VIII antibody (clone 2C11). The cDNA will be reverse transcribed using an oligo-dT primer. After eliminating the template RNA with RNAse H, the cDNA will be used as the PCR template. An oligonucleotide primer pair was designed based on the consensus constant region for human IgG4 heavy chain: IgG4-forward 5′-agagagaattccttccaccaa-3′ (SEQ ID NO: 22) and IgG4-reverse 5′-acccacactagttttacccagagaca-3′ (SEQ ID NO: 23). At the 5′ end, the primers contain an EcoRI or a SpeI site (shown in italics). The stop codon “tga” at the end of the CH3 domain was not included in the reverse primer. The amplified PCR fragment (CH1-hinge-CH2-CH3) will be cloned into the pBSSK vector using the EcoRI and SpeI restriction sites. In the resulting vector, the 36 bp hinge region agtccaaatatggtcccccatgcccatcatgcccag (SEQ ID NO: 24)) of the hIgG4 will be deleted using the site-directed mutagenesis kit (Strategene) per the manufacturer's instructions.

pBSSK-MOG₃₅₋₅₅

For testing BAIT in a mouse model of MS, the oligonucleotide primers were designed based on the murine MOG₃₅₋₅₅ encoding sequence (MEVGWYRSPFSRVVHLYRNGK (SEQ ID NO: 25)): MOG₃₅₋₅₅-forward 5′-agcagactagtatggaggtgggt-3′ (SEQ ID NO: 26) and MOG₃₅₋₅₅-reverse 5′-attatgcggccgccttgccatttcggt-3′ (SEQ ID NO: 27). The forward and reverse primers contain a SpeI and a NotI site at the 5′ and 3′ ends, respectively. A vector encoding murine MOG (kind provided by Dr. Joan Goverman, University of Washington) will be used as the PCR template. The amplified fragment will be cloned into the pBSSK vector using the restriction sites SpeI and NotI. Note that, human MOG₃₅₋₅₅, which differs from murine MOG₃₅₋₅₅ only by a proline substitution at position 42, is encephalitogenic in DR2 transgenic mice²⁰. A vector containing human MOG₃₅₋₅₅ sequence will be generated based on the pBSSK-mMOG₃₅₋₅₅ using the above mentioned site-directed mutagenesis, for future clinical testing.

pBSSK-svCD20-hIgG4-MOG₃₅₋₅₅, -OVA₃₂₃₋₃₃₉, and -GFP

Next, the IgG4 fragment will be cut out from the pBSSK-hIgG4 vector and ligated into the pBSSK-svCD20 vector using the EcoRI and SpeI restriction sites. The resulting vector is termed pBSSK-svCD20-hIgG4. To construct pBSSK-svCD20-hIgG4-mMOG₃₅₋₅₅, the murine MOG fragment will be cut out through the SpeI and NotI sites, followed by ligating the fragment into pBSSK-svCD20-hIgG4 vector digested with the same enzymes.

The pBSSK-svCD20-hIgG4-OVA₃₂₃₋₃₃₉ and pBSSK-svCD20-hIgG4-GFP will be constructed based on the pBSSK-svCD20-hIgG4 vector. The OVA₃₂₃₋₃₃₉ and GFP with flanking SpeI and NotI sites will be PCR amplified from relevant vectors. The oligonucleotide primer pair for OVA₃₂₃₋₃₃₉ is: forward 5′-agcgcactagtaagatatctcaagct-3′ (SEQ ID NO: 28), reverse 5′-attatgcggccgcgcctgcttcattga-3′ (SEQ ID NO: 29). The primer pair for GFP is: forward 5′-actccactagtatggtgagcaa-3′ (SEQ ID NO: 30) and reverse 5′-attatgcggccgccttgtacagctcgt-3′ (SEQ ID NO: 31). The PCR products will be digested with SpeI and NotI restriction enzymes and ligated into the SpeI/NotI digested pBSSK-svCD20-hIgG4.

In addition, based on the above created vectors, we will also generate pBSSK-hIgG4-MOG₃₅₋₅₅ (MOG₃₅₋₅₅IgG, termed MOG-IgG herein and pBSSK-hIgG4-GFP (Ig-GFP), to serve as additional controls that lack the svCD20 component. A summary of the subcloning strategy is shown in Table 3 below.

Expression and Purification of svCD20-hIgG4-MOG₃₅₋₅₅ Tolerogen (BAIT)

For mammalian expression, the insert containing svCD20-IgG4-MOG₃₅₋₅₅ will be cut out from the pBSSK-svCD20-hIgG4-MOG₃₅₋₅₅ vector by HindIII and NotI digestion. The obtained fragment will then be ligated into HindIII/NotI digested pSecTag2 vector. The resulting vector is pSec-svCD20-hIgG4-MOG₃₅₋₅₅-Tag2. The recombinant protein expressed in the pSecTag vector will be fused on its C-terminus with a c-Myc epitope and six tandem histidine tag (SEQ ID NO: 32). The entire insert will be sequenced to ensure that the sequence and the coding frame are correct. The control expression vectors pSec-svCD20-hIgG4-OVA₃₂₃₋₃₃₉-Tag2 and pSec-svCD20-hIgG4-GFP-Tag2 will be generated similarly.

CHO-K1 cells (ATCC) will be used to express the BAIT and other control fusion proteins. For expression of BAIT, CHO-K1 cells will be transiently transfected with svCD20-IgG4-MOG₃₅₋₅₅ using standard calcium phosphate method. Stable integrants will be selected for zeocin resistance. The supernatant from the zeocin resistant clones will be screened for recombinant fusion protein expression using a dot blot protocol for detection of the C-terminus fused c-Myc tag in the fusion protein.

TABLE 3
Summary of subcloning strategy for BAIT molecules

| BAIT | | Primer (SEQ ID NOS 20-23 and 26-31, | Subcloning |
|---|---|---|---|
| Domain | Template | respectively, in order of appearance) | Site |
| svCD20 | pCG-H-αCD20 | Forward: 5′-agaggaagcttatggctcaggttca-3′ | HindIII, EcoRI |
| | | Reverse: 5′-agagcgaattccttcagctccagct-3′ | |
| hIgG4 | First-strand | Forward: 5′-agagagaattccttccaccaa-3′ | EcoRI, SpeI |
| | cDNA | Reverse: 5′-acccacactagttttacccagagaca-3′ | |
| Antigen | MSCV-MOG₁₋₁₂₉ | MOG₃₅₋₅₅-F 5′-agcagactagtatggaggtgggt-3′ | SpeI, NotI |
| | | MOG₃₅₋₅₅-R 5′-attatgcggccgccttgccatttcggt-3′ | |
| | pBSSK-OVA | OVA₃₂₃₋₃₃₉-F 5′-agcgcactagtaagatatctcaagct-3′ | |
| | | OVA₃₂₃₋₃₃₉-R 5′-attatgcggccgcgcctgatcattga-3′ | |
| | MSCV-IRGFP | GFP-F 5′-actccactagtatggtgagcaa-3′ | |
| | | GFP-R 5′-attatgcggccgccttgtacagctcgt-3′ | |

After the high efficiency clones are identified, the cells will be adapted for growth in suspension, and the fusion protein in the supernatant from large-scale suspension culture will be purified through affinity chromatography on Ni-NTA agarose columns (Qiagen, Valencia, Calif., USA) per the manufacturer's instruction. Purification will be carried out using a Bio-Rad HPLC unit. The fractions with the highest target fusion protein will be monitored by the absorbance at OD280 and evaluated by sodium dodecyl sulfate polyacrylamide gel electrophoresis (SDS-PAGE) followed by Coomassie blue R-250 staining, Western blot analysis and mass spectrometry (see below). Using the same technique, fusion proteins comprised with FVIII domains fused together with different portions of a murine IgG1 heavy chain on a milligram scale per 100 ml culture supernatant can be produced.

Adherent CHO cells plated in 6-well plates were transfected with pSec-svCD20-Ig-MOG₃₅₋₅₅ (BAIT-MOG), pSec-svCD20-Ig-OVA323-335 (BAIT-OVA), or pSec-svCD20-Ig-GFP (BAIT-GFP). 48 hours later, the supernatant or cell lysate samples were collected and separated in a 4-12% PAGE gel, and blotted with anti-human IgG or GFP. The expected size for the monomer BAIT fusions are about 67, 67, and 92 KD for BAIT-MOG, BAIT-OVA, and BAIT-GFP, respectively. The major specific bands detected using anti-human IgG or anti-GFP antibodies were smaller than expected. This indicated to us that the expressed proteins were not full length. Moreover, GFP part was only detectible in the cell lysate but not in the supernatant sample. These results suggested possible inappropriate folding of the expressed BAIT fusions, which may lead to intracellular degradation and inefficient protein export.

To address this potential problem, we designed BAIT fusions wherein the antigen component (e.g., MOG or FVIII) is downstream of the svCD20 and upstream of the IgG moiety, as described in more detail below.

### GMP Production of BAIT Fusion Proteins

For clinical quality GMP production of the BAIT fusion proteins, the stop codon will be introduced at the end of the antigen module in the above pSecTag2 vectors. Thus, c-Myc and six tandem Histidine tags (SEQ ID NO: 32) will not be part of the fusion protein. During the purification step, a Protein A column will be used instead of the Ni-NTI column.

### Characterization of BAIT Proteins

Molecular Weight

The native molecular mass of the BAIT fusion protein will be estimated using non-denaturing PAGE gel electrophoresis. Purified BAIT fusion protein (0.1 μg) will be mixed in the loading buffer (1 mM sodium phosphate, pH7.0, and 50 mM NaCl) and separated in 7.5% non-denaturing polyacrylamide gel according to the standard protocol. The native molecular mass of the fusion protein will be estimated by comparing to the protein standards.

Antibody Binding and Specificity

The binding capacity of the fusion protein will be assayed with the Raji human B-cell line, which we know to be positive for CD20. Purified fusion protein BAIT-GFP will be added to Raji cells (10⁶) at concentrations from 0.1-20 μg/ml for 1 hour at 4° C. After washing 3× in PBS, the cells will be analyzed by flow cytometry for GFP fluorescence. Background staining will be determined by using Ig-GFP fusion protein, which does not contain the svCD20 domain.

To determine the B-cell specific binding of the fusion protein, splenocytes from human CD20 transgenic mice, as well as human PBMC, will be used. Purified BAIT-GFP will be added to the cells (10⁶) at standard concentrations for 1 hour at 4° C. After washing, the percentage of GFP positive cells among different populations of cells will be determined by flow cytometry. The gating strategy will be: B cells (CD19⁺), macrophages (F4/80⁺), dendritic cells (CD11c⁺), neutrophils (Ly-6G⁺), non-leukocytes (CD45⁻), T cells (CD4⁺, CD8⁺CD3⁺), platelets/megakaryocytes (CD41⁺).

### Transduction of B Cells

For tolerogenic antigen presentation, surface bound fusion protein needs to be internalized by the B cells. To determine whether the fusion protein can be internalized by the B cells, purified BAIT-GFP will be added to the splenic B cells from human CD20 transgenic mice and incubated at 37° C. for 30 minutes to six hours. After washing, the cells will be treated with trypsin for 5 min at room temperature to remove surface bound GFP fusion protein and the fluorescence intensity analyzed by flow cytometry. Background fluorescence will be determined by incubating the cells with control Ig-GFP. Transduction efficiency between different populations of splenic B cells will be compared for FO (CD19⁻CD23^{high}CD21^{low}), MZ (CD19⁻CD23^{low}CD21^{high}), B1 B (CD19⁺CD23^{low}IgM^{high}CD93⁻CD43⁺), and plasmablasts (CD19⁺IgM^{high}VLA4^{low}CD138⁺).

### In Vitro T Cell Proliferation Assay and Assay for Anergy/Suppression

B-cell transduction by the BAIT fusion protein will also be examined as follows in 96 well flat-bottomed plates in vitro. Irradiated (1500 rad) splenic B cells (2×10⁵/100 μl) from human CD20 transgenic mice (C57BL/6 background) will be pulsed with BAIT-MOG₃₅₋₅₅ (at concentrations based on FACS results) or BAIT-OVA₃₂₃₋₃₃₉ at 37° C. for 4 hrs. CD4+ T cells (0.5×10⁵/100 μl) from 2D2 (MOG-specific TCR transgenics) or lymph node cells (1 ×10⁵/100 μl) from MOG₃₅₋₅₅/CFA immunized mice will be added to each well. MOG₃₅₋₅₅ and OVA₃₂₃₋₃₃₉ will be used as positive and negative controls, respectively. The response of OT-II OVA specific TCR transgenic cells to BAIT-OVA₃₂₃₋₃₃₉ will be similarly tested. After 48 hrs at 37° C., the cells will be pulsed with 1 μCi of 3H-thymidine for an additional 12-16 hrs, and 3H-thymidine incorporation will be determined by standard methods per previous publications²¹. The CPM obtained from the wells that contain no BAIT-MOG₃₅₋₅₅ or OVA peptide will be subtracted to obtain delta CPM.

If BAIT-MOG₃₅₋₅₅ fails to stimulate 2D2 T cells, it is to be determined whether they have been anergized or become suppressive in subsequent two-stage assays. Basically, for “anergy”, BAIT pre-cultured T cells (from 24-well cultures) will be washed and then “challenged” with optimal doses of MOG₃₅₋₅₅ and freshly irradiated spleen cell APC and assayed for proliferation and cytokine production (IFNγ, IL-4).

To assay for suppression (and Treg induction), BAIT pre-cultured cells (from 2D2, MOG-immunized or naïve C57Bl/6 mice) will be added to CFSE-labeled 2D2 splenocytes in various ratios (keeping target 2D2 T cells constant) and CFSE dilution will be measured using flow cytometry. In addition, 2D2-FoxP3GFP spleen cells can be used in the primary culture to determine whether FoxP3+ Tregs are increased upon culture with BAIT-MOG₃₅₋₅₅.

### In Vivo Tolerance Assay

A long-term goal for this project is to use BAIT-MOG₃₅₋₅₅ as a therapy in EAE, a step towards its use in clinical trials. Based on the preliminary in vitro analyses above, C57Bl/6 mice will be treated either prophylactically and then immunized with MOG peptide in CFA+pertussis or (more importantly) treated after symptoms appear in our standard EAE protocol, as outlined in the next aim.

### Other BAIT Proteins

A single chain anti-CD19 mAb sequence can be cloned at the N-terminus of the fusion to replace the svCD20 sequence, as described above. While it is possible that the BAIT MOG₃₅₋₅₅ could induce anergy or suppression, an increase in FoxP3+ cells with the 2D2 cells is expected. Regulatory T cell suppression assays are well established in the art. The BAIT construct can be modified with an MBP peptide for translation using the Ob.1A12 T cell clone from an MS patient. Further mutations of the IgG4 heavy chain protein can be made to prevent FcγR binding activity, adjust the position of each component in the BAIT molecule, and/or eliminate the c-myc and six tandem histidine tag (SEQ ID NO: 32) in the final fusion product.

### Example 2

Determination of the Tolerogenic Effect of the BAIT Fusion Protein Used in Combination With/Without a Selective B-Cell Depleting Agent in A Mouse Model for MS.

### Overall Strategy

The present invention establishes effective novel biotherapeutics for MS, which will not only alleviate disease symptoms, but also will address the underlying autoimmune problem. BAIT-MOG₃₅₋₅₅ will be tested in combination with a partial B-cell depletion agent, IgG1 anti-mouse CD20 mAb, which spares MZ B cells and favors tolerance. It is to be determined whether BAIT-MOG₃₅₋₅₅ may work by itself (and better than generic MOG-IgG) or in combination with partial B-cell depletion as an effective therapy in a mouse model for MS, EAE. Further, the BAIT tolerogenic fusion protein therapy in MS patient's PBMC reconstituted Rag2^{-/-} mice will be tested. Initially, human CD20 transgenic mice of C57BL/6 background (hCD20 Tg; provided by Dr. Mark Shlomchik under an approved MTA) are used as both donors and recipients in in vivo experiments. The hCD20 transgene expressed on B-cells is recognized by the svCD20 in the fusion protein (and verified above). Importantly, these B cells still express endogenous mouse CD20, which allow for depletion with IgG1 anti-mouse CD20. For the disease induction, the active MOG₃₅₋₅₅ induced chronic EAE model is used in this study. The disease course will be evaluated daily with the standard 0-5 scoring system used in previous publications^{21,23,24}. In addition, histology evaluation (see below) on the spinal cord tissue will be performed at various time points to examine the potential CNS repair and remyelination that may progress upon tolerogenic therapy.

### To Determine the Effect of Tolerogenic Fusion Protein BAIT in a Mouse Model of MS

It is to be determined whether administration of BAIT-MOG₃₅₋₅₅ alone is tolerogenic and has a therapeutic effect on MOG₃₅₋₅₅-induced EAE. In this experiment, 6-8-week old female hCD20 Tg mice (n=8) will be actively induced for EAE at day 0 in a standard protocol (subcutaneous injection into the flank and the base of tail) with 100 μg MOG₃₅₋₅₅ peptide emulsified in CFA containing 4 mg/ml of Mycobacterium tuberculosis H37Ra (DIFCO, Detroit, Mich.). On day 0 and 48 h later, the mice will also receive 200 ng of pertussis toxin (Sigma-Aldrich) in 0.2 ml PBS intraperitoneally.

Starting either at day −7 (prophylactic) or day+10 (therapeutic), the mice will be i.v. injected daily for 5 days with 100 μg of BAIT-MOG₃₅₋₅₅, 100 μg of BAIT-OVA₃₂₃-339 (specificity control), MOG-IgG or PBS (vehicle control). The disease course and histopathology will be evaluated as described below. On day 45, mice from the treatment and control groups will be perfused with 0.9% saline followed by cold 4% paraformaldehyde and spinal cords will be removed and post-fixed in 4% paraformaldehyde and section stained with luxol fast blue/periodic acid-Schiff-hematoxylin, and analyzed by light microscopy to assess demyelination and inflammatory lesions. Inflammation will be scored as: 0=no inflammation, 1=inflammatory cells only in leptomeninges and perivascular spaces, 2=mild inflammatory infiltrate in spinal cord parenchyma, 3=moderate inflammatory infiltrate in parenchyma, 4=severe inflammatory infiltrate in parenchyma. Demyelination will be scored as: 0=no demyelination, 1=mild demyelination, 2=moderate demyelination, 3=severe demyelination.

To examine the potential effects of BAIT-MOG₃₅₋₅₅ on CNS repair and remyelination, another cohort of hCD20 Tg mice (n=5) will first be immunized with MOG₃₅₋₅₅/CFA/Pertussis toxin for disease induction. The administration of the BAIT-MOG, BAIT-OVA or PBS will be initiated during the peak of the disease (˜day 21). The mice will be scored daily and at the end of the experiment (45 days after immunization), spinal cord histopathology will be performed as described above. CNS inflammation and demyelination status will be compared between the BAIT-MOG₃₅₋₅₅ group and the control groups.

### To Determine the Effect of Selective FO B-Cell Depletion in a Mouse Model for MS

It has been previously found that IgG isotypes of anti-mouse CD20 mAbs (provided by Biogen Idec) have differential effects in terms of B-cell depletion. Thus, while IgG2a anti-CD20 mAb mediates complete B-cell depletion, the IgG1 anti-CD20 largely spares MZ B cells and favors tolerance⁵. The effect of IgG1 versus IgG2a anti-CD20 mAb in the MOG-induced mouse model for MS independent of BAIT treatment are compared. Six-8-week female hCD20 Tg mice (n=8) will be actively induced for EAE as above. The mice will be treated with IgG1 anti-CD20, IgG2a anti-CD20, or PBS (vehicle) either at day −7 or at day 7. The disease course will be evaluated daily after the immunization using the scoring system mentioned above. In addition, at the end of the experiment (usually d45), spinal cords will be examined for evidence of demyelination as above.

### To Determine the Effect of Combination Therapy (B-Cell Depletion+BAIT Treatment) in a Mouse Model for MS

Finally, the strategy of combining a selective FO B-cell depletion with the BAIT-MOG₃₅₋₅₅ therapy will be evaluated in MOG-induced EAE. Female 6-8-week old hCD20 Tg mice (n=8) will be actively induced for EAE at day 0 with MOG₃₅₋₅₅/CFA/Pertussis toxin. Two weeks before the disease induction, the mice will be injected i.v. with 250 μg (˜10 mg/kg) of IgG1 anti-CD20. Starting on day+7, the mice will be injected i.v. daily for 5 days with 100 μg BAIT-MOG₃₅₋₅₅, 100 μg BAIT-OVA₃₂₃₋₃₃₉ (specificity control), MOG-IgG or PBS (vehicle control). The disease course and CNS histopathology will be followed as described in sections above.

### Expected Outcomes, Potential Challenges and Alternative Strategies

This study focuses on targeting B cells for tolerogenic antigen presentation and inducing tolerance to CNS antigen-specific CD4+ T cells. Accordingly, MOG₃₅₋₅₅ peptide induced mouse EAE was chosen to evaluate the efficacy of the proposed therapy. The effect of B-cell depletion using different isotypes of anti-mouse CD20 has also been previously reported⁵ (Zhang A H, et al. Blood. 2011). With a single dose of anti-CD20 mAb, the peak of B-cell depletion occurs at around 2 weeks. After that, the B-cell repertoire will slowly and gradually recover. By the time of initiation of the BAIT-MOG₃₅₋₅₅ injection in the combination therapy strategy, newly emerged naïve resting B cells and those remaining MZ B cells will be ideal targets for the B-cell specific tolerogenic fusion protein. The BAIT treatment is expected to be more effective than MOG-IgG (or as effective at lower doses) and the combined therapy with IgG1 anti-CD20 depletion may be even more effective in treating EAE therapeutically. If B-cell surface CD20 is not as efficiently endocytosed by B cells as needed for processing and presentation, the svCD20 in the fusion will be replaced by an anti-CD19 single chain antibody. However, since human CD19 transgenic mice were reported to have severe defects in early B-cell development⁵, an anti-mouse CD19 single chain sequence will be used in the fusion and wild type mice will be used for the proof-of-principle EAE experiments.

### Example 3

BAIT fusions were designed and constructed with the configuration:
B cell-targeting molecule-antigen-IgG4H
   as described in more detail below and illustrated in FIG. 3B.

### Construction of BAIT Expression Vectors

Two pFuse expression vectors encoding svCD20-FVIII₂₁₉₁₋₂₂₁₀ and svCD2O-OVA₃₂₃₋₃₃₉ were constructed. Additionally, a pFuse expression vector encoding svCD19-MOG₃₅₋₅₅-hIgG4 was also generated. The BAIT protein based on the anti-mouse svCD19 was abbreviated as mBAIT, to distinguish those fusions based on anti-human CD20 scFv.

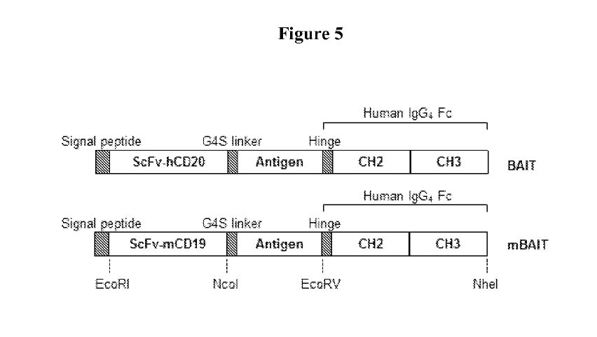

FIG. 5 illustrates BAIT and mBAIT expression cassettes differing in the B-cell targeting module. The former uses anti-human CD20 single chain variable fragment (svCD20), which is specific for human B cells or human CD20 transgenic mouse B cells. The latter is specific for mouse B cells by using anti-mouse CD19 scFv (msvCD19) for targeting. For facilitating cloning and protein expression, a commercially available hIgG4 fusion protein expression vector, pFuse-hIgG4-Fc2 (Invivogen), was used. The ScFv-Antigen fragment was cloned into the pFuse-hIgG4-Fc2 vector between the IL-2 signal sequence and the human IgG4 hinge using the EcoR1 and EcoRV restriction site. The svCD20-antigen or msvCD19-antigen cDNA fragment was codon optimized for expression in CHO cells (see SEQ ID NOs 2 and 5) and synthesized by GenScript. BAIT and mBAIT expression vectors containing other antigens, for example, FVIII C2 and A2 domains,can be generated using the same restriction sites flanking the antigen component. BAITs containing OVA₃₂₃₋₃₂₉ or OVA were used as the antigen specificity control.

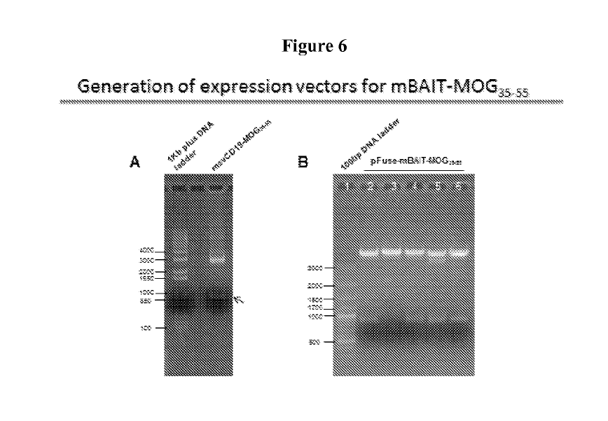

Furthermore, expression vectors for mBAIT-MOG₃₅₋₅₅ were generated. The EcoRI and EcoRV/BamHI flanked svCD19-MOG₃₅₋₅₅ DNA fragment was synthesized by GenSript. The svCD19-MOG₃₅₋₅₅ insert was cut out from the intermediate pUC57-svCD19-MOG₃₅₋₅₅ vector using EcoRI and BamHI, and then ligated into the EcoRI/BglII digested pFuse-hIgG4-Fc2 vector. Subsequently, after confirming the size of the insert, the colonies for the correct expression vectors were screened by restriction analysis, as illustrated by FIG. 6.

### Example 4

Generation of BAIT-FVIII₂₁₉₁₋₂₂₁₀ and BAIT-OVA₃₂₃₋₃₃₉ Fusion Proteins

The BAIT-FVIII₂₁₉₁₋₂₂₁₀ and BAIT-OVA₃₂₃₋₃₃₉ fusion proteins were expressed by the expression vectors described above. BAIT-FVIII₂₁₉₁₋₂₂₁₀ and BAIT-OVA₃₂₃₋₃₃₉ fusion protein expression and secretion were successful in transiently transfected CHO cells.

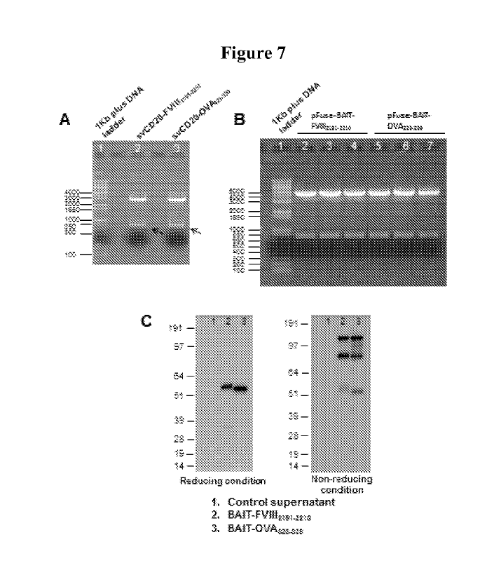

As illustrated by FIG. 7, the inserts were analyzed and confirmed by restriction analysis (FIG. 7A, FIG. 7B), gel purified and cloned into EcoRI/BgIII digested pFuse-hIgG4-Fc2 expression vectors. Following restriction analysis using EcoRI and NcoI to screen for the vectors having the correct inserts, the selected vectors were purified for further transfection and protein expression analysis.

Specifically, Western blot analysis was performed to verify the expression (FIG. 7C). CHO cells were transfected with either pFuse-BAIT-FVIII₂₁₉₁₋₂₂₁₀ or BAIT-OVA₃₂₃₋₃₃₉ plasmid DNA. The supernatant were collected 48 hrs after transfection and protein expression was analyzed by Western blot in NuPage 4-12% Bis-Tris gel. Under reducing condition, only one major band was detected at size of ˜56 KD for both of the proteins. Majority of the BAIT fusion proteins were in the form of polymers, as revealed by Western blot with non-reducing condition. The blotting antibody used was monoclonal anti-human IgG (λ, chain specific) and HRP Rabbit anti-mouse IgG (H+L).

### Example 5

### Generation of Stably Transfected CHO Cell Lines

This example demonstrates that stably transfected CHO cell lines for BAIT-FVIII₂₁₉₁₋₂₂₁₀ and BAIT-OVA₃₂₃₋₃₃₉ fusion proteins were established, and BAIT protein expression in these lines were verified.

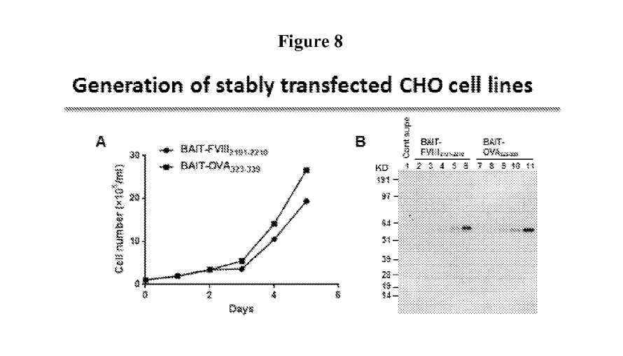

Adherent CHO cells in 6-well plate were transfected with 2.5 μg of either pFuse-BAIT-FVIII₂₁₉₁₋₂₂₁₀ or BAIT-OVA₃₂₃₋₃₃₉ plasmid DNA using lipofectamine LTX reagents. The transfected cells were selected with 500 μg/ml zeocin for 20 days. The selected stably transfected CHO cells were then adapted to suspension culture condition using serum free FreeStyle CHO medium containing 1× GlutaMax, 0.5× pen-strep and 100 μg/ml zeocin, in 37° C., at 8% CO2 and with shaking at 125 rpm. The cells were plated at 1×10E5/ml in 250 ml polycarbonate disposable flasks with total volume of 80 ml. The supernatant samples were collected every day from the suspension cultures, and cell number counted. The cell growth curve of FIG. 8A shows that the viable cell were more than 95% at all data points.

Western blot analysis was performed on the supernatant samples from the stably transfected CHO cells in NuPage 4-12% Bis-Tris gel under reducing condition. In FIG. 8B, Lane 2-6 and 7-11 were day 1-5 supernatant samples from BAIT-FVIII₂₁₉₁₋₂₂₁₀ and BAIT-OVA₃₂₃₋₃₃₉ stable lines, respectively. The levels of BAIT protein expression accumulated over the days of the suspension culture, and only one single band at ˜56 KD was detected for both the fusion proteins. The blotting antibody used was monoclonal anti-human IgG (λ, chain specific) and HRP Rabbit anti-mouse IgG (H+L).

### Example 6

### Determination of Efficacy of BAIT Fusion Proteins

Once the expression of each BAIT fusion protein is verified in transfected CHO cells, the BAIT fusion protein is purified according to established protocols.

The B-cell specific binding in vitro is examined using human PBMC B cells or splenic B cells from human CD20 transgenic mice for BAITs, and C57Bl/6 mouse splenic B cells for mBAIT. Subsequent in vitro uptake/presentation will be analyzed by T cell proliferation assay utilizing an in house generated FVIII₂₁₉₁₋₂₂₁₀ specific human T cell line, and T cells from OT-II and 2D2 transgenic mice for BAIT-OVA₃₂₃₋₃₃₉ and mBAIT-MOG₃₅₋₅₅, respectively.

Additionally, the efficacy of BAIT-MOG₃₅₋₅₅ for multiple sclerosis is examined using human CD20 transgenic mice. Moreover, the efficacy of mBAIT-MOG₃₅₋₅₅ and mBAIT-A2/mBAIT-C2 for multiple sclerosis and hemophilia A with inhibitor, respectively is examined in mice model.

### Example 7

### In Vitro B-Cell Specific Binding of the BAIT Fusion Proteins BAIT_{hCD20-FVIII2191-2210}

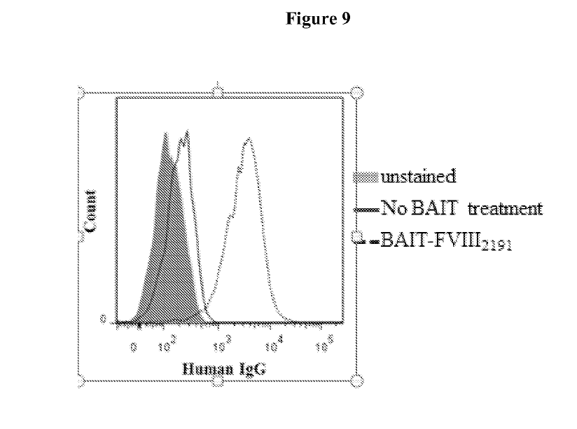

The binding capacity of a BAIT fusion protein (of BAIT_{hCD20}-FVIII₂₁₉₁₋₂₂₁₀) was assayed using the Raji human B-cell line, which is known to be positive for CD20. Raji cells (5×10̂5) were incubated with 1 μg of purified BAIT_{hCD20}-FVIII₂₁₉₁₋₂₂₁₀ for 1 hour at 37° C. The cells were then stained with APC anti-human IgG, which recognizes the human IgG4 Fc region of the BAIT fusion protein. After washing 3 times with PBS buffer, the cells were analyzed by flow cytometry. The cells were gated on live singlet. The data show that the BAIT fusion protein bound to the CD20+ B cells. See FIG. 9.

The ability of the BAIT fusion protein to bind B cells of human PBMC B cells also was performed. Human PBMC were incubated with biotinylated BAIT_{hCD20}-FVIII₂₁₉₁₋₂₂₁₀ or left untreated as control. After incubation, the cells were blocked with human FcR blocking reagent (Miltenyi Biotech) for 15 minutes at 4° C. The cells were extracellularly stained with FITC-conjugated streptavidin, APC eFluo780 (viability dye) and CD19-APC, and then intracellularly stained with PE-conjugated streptavidin. After washing, the percentage of extracellular and intracellular cells among different populations of cells was determined by flow cytometry. Overlap dotplots (not shown) revealed that only CD19+ B cells were FITC-streptavidin positive, which indicates B-cell specific binding. Compared to incubation at 4° C., a significant number of B cells were also PE-streptavidin positive following 1 hour incubation at 37° C., suggesting B cell uptake of the BAIT fusion protein.

Confocal images of cells treated with biotinylated BAIT_{hCD20}-FVIII₂₁₉₁₋₂₂₁₀ for 60 min at 37° C. and stained as described above were obtained (not shown). The non-overlapping extracellular and intracellular fluoresence signal indicates the B cell binding and uptake of the BAIT fusion protein.

### Example 8

### Effect of the BAIT Fusion Protein BAIT-FVIII₂₁₉₁₋₂₂₁₀ on the Proliferation Response of Specific CD4+ Effector T Cells.

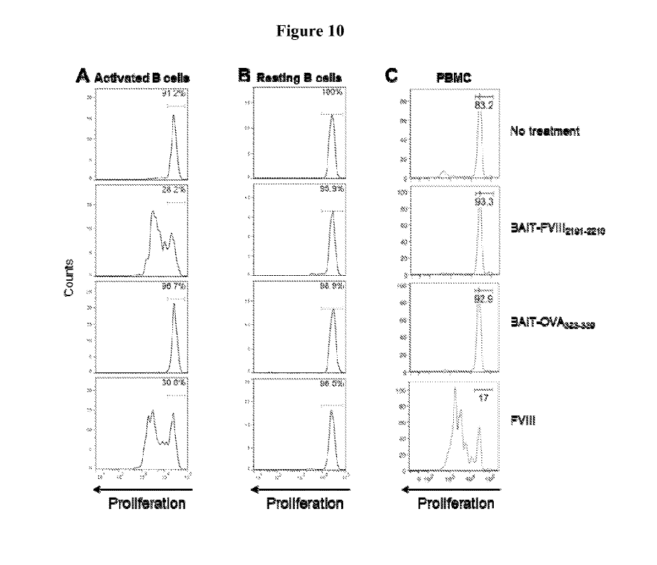

The in vitro uptake/presentation of BAIT fusion protein BAIT-FVIII₂₁₉₁₋₂₂₁₀ was analyzed by T cell proliferation assay utilizing an in house generated FVIII₂₁₉₁₋₂₂₁₀ specific human T cell line. Human B cells from a HLA DR1/DR2 donor were purified using anti-CD19 magnetic beads (Miltenyi) and activated with 2 μg/ml CD40L plus 10 ng/m IL-4 for 3 days. The activated B cells were then co-cultured with proliferation dye eFluor 450 labeled 17195 T effectors at the ration of 5:1, in the absence or presence of 1 μg/ml of either BAIT_{hCD20}-FVIII₂₁₉₁₋₂₂₁₀, BAIT_{hCD20}-OVA₃₂₃₋₃₃₉, or recombinant FVIII. After three days, the proliferation status of 17195 T effectors were evaluated by flow cytometry based on the dilution of the eFluor 450 fluorescence signal. See FIG. 10. This experiment shows that the FVIII₂₁₉₁₋₂₂₁₀ epitope of the BAIT fusion was appropriately processed and presented to specific T cells by activated human B cells.

The effect of BAIT fusion protein on the proliferation response of T cells to resting B cells also was tested. Purified human B cells from a HLA DR1/DR2 donor were directly co-cultured with the labeled 17195 T effectors at the ration of 5:1, in the absence or presence of 1 μg/ml of either BAIT_{hCD20}-FVIII₂₁₉₁₋₂₂₁₀, BAIT_{hCD20}-OVA₃₂₃₋₃₃₉, or recombinant FVIII. The proliferation response of 17195 T effectors was evaluated as above. See FIG. 10. This experiment shows that resting B cells pulsed with BAIT_{hCD20}-FVIII₂₁₉₁₋₂₂₁₀ or FVIII did not support the proliferation response of specific T cells. The absence of T cell proliferation suggests the antigen presentation by resting B cells favors tolerance by inducing T cell anergy or population of cells with suppressing activity. (An unlikely alternative explanation for this result is that resting B cells were unable to uptake and present the antigen delivered by BAIT.)

Human PBMC from a HLA DR1/DR2 donor were co-cultured with the labeled 17195 T effectors at the ratio of 20:1, in the absence or presence of 5 μg/ml of either BAIT_{hCD20}-FVIII₂₁₉₁₋₂₂₁₀ or BAIT_{hCD20}-OVA₃₂₃₋₃₃₉, or 1 μg/ml of recombinant FVIII. The proliferation response of 17195 T effectors was evaluated as above. See FIG. 10. This experiment shows that human PBMC pulsed with BAIT_{hCD20}-FVIII₂₁₉₁₋₂₂₁₀ did not support the proliferation response of specific T cells. As discussed above, this result indicates that delivery of antigen by using a BAIT fusion protein favors tolerance induction.

### Example 9

### In Vivo Efficacy of FVIII BAIT to Induce Tolerance to FVIII in Naive Mice

To show that FVIII BAIT fusion protein is tolerogenic in FVIII naïve mice, transgentic FVIII knock-out mice will be administered a FVIII BAIT fusion protein, and then administered FVIII, and the immune response to FVIII will be assessed.

For example on Day 0, Group 1 will be intravenously administered 10 μg mBAIT-FVIII C2, Group 2 will be injected with 10 μg mBAIT_-OVA, Group 3 will be injected with 50 μg mBAIT-FVIII C2, and Group 4 will be injected with 50 μg mBAIT-OVA. Then, on Days 7, 14 and 21, the mice will be challenged with FVIII (1 μg in 100 μl PBS; i.v.). On day 28, blood will be drawn to determine the immune response to FVIII. In addition, the mice will be euthanized to obtain spleen for lymphocyte proliferation and ELIspot assays.

It is anticipated that Group 2 and Group 4 will not show tolerance to FVIII. Group 1 and Group 3 may demonstrate tolerance to FVIII, possibly in a dose-dependent manner.

### Example 10

### In Vivo Efficacy of FVIII BAIT to Induce Tolerance to FVIII Immunized Mice

To show that FVIII BAIT fusion protein is tolerogenic for subjects immunized with FVIII, FVIII knock-out mice will be immunized with FVIII, administered a FVIII BAIT fusion protein, and the immune response to FVIII will be assessed before and after BAIT treatment.

For example, mice will be administered rFVIII (1 μg in 100 μl PBS; i.v.) at Days 0, 7, and 14. On Day 21, blood will be drawn to determine the immune response to FVIII. Mice will be randomly divided into four groups. On Days 28, 35, and 42, Group 1 will be intravenously administered 10 μg mBAIT-FVIII C2, Group 2 will be intravenously administered 10 jig mBAIT_-OVA, Group 3 will be intravenously administered 50 μg mBAIT_-FVIII C2, and Group 4 will be intravenously administered 50 μg mBAIT_-OVA. On Days 35, 42, and 49, blood will be drawn to determine the immune response to FVIII. On Day 49, mice will be administered rFVIII (1 μg in 100 μl PBS; i.v.). On Day 56 blood will be drawn to determine the immune response to FVIII. In addition, the mice will be euthanized to obtain spleen for lymphocyte proliferation and ELIspot assays.

It is anticipated that Group 2 and Group 4 will not show tolerance to FVIII. Group 1 and Group 3 may show tolerance to FVIII, possibly in a dose-dependent manner

### Example 11

In Vivo Efficacy of MOG BAIT to Induce Tolerance in EAE Mice Induced with MOG₃₅₋₅₅ Peptide/CFA/PT

The efficacy of MOG BAIT fusion protein against multiple sclerosis will be shown in mice induced with experimental autoimmune encephalomyelitis (EAE).

For example, mice will be induced with EAE using MOG35-55 peptide emulsified in CFA (Day 0). Also on Day 0 and on Day 2, mice will be intraperitoneally administered Pertussis toxin (PT) (50 ng in 200 μl PBS). Clinical signs of EAE will be evaluated daily starting on Day 7. EAE mice will be randomly assigned to four groups. On Days 7, 14, 21, and 28, Group 1 will be intravenously administered 10 μg mBAIT_-MOG₃₅₋₅₅, Group 2 will be intravenously administered 10 μg mBAIT_-OVA, Group 3 will be intravenously administered 50 μg mBAIT-MOG₃₅₋₅₅, and Group 4 will be intravenously administered 50 μg mBAIT_-OVA. On Day 35, the mice will be euthanized to obtain spinal cord samples for myelin immunohistochemistry staining.

It is anticipated that Groups 1 and 3 will exhibit protection from EAE, possibly in a dose-dependent manner

### REFERENCES

1 Hauser S L, Waubant E, Arnold D L, et al. 2008. B-cell depletion with rituximab in relapsing-remitting multiple sclerosis. N Engl J Med. 358:676-88.

2 Reichardt P, Dornbach B, Rong S, et al. 2007. Naïve B cells generate regulatory T cells in the presence of a mature immunologic synapse. Blood. 110:1519-1529.

3 Matsushita T, Yanaba K, Bouaziz J D, et al. 2008. Regulatory B cells inhibit EAE initiation in mice while other B cells promote disease progression. J Clin Invest. 118: 3420-30.

4 Yu S, Dunn R, Kehry M R, Braley-Mullen H. 2008. B cell depletion inhibits spontaneous autoimmune thyroiditis in NOD.H-2h4 mice. J Immunol. 180:7706-13.

5 Zhang A H, Skupsky J, Scott D W. 2011. Effect of B-cell depletion using anti-CD20 therapy on inhibitory antibody formation to human FVIII in hemophilia A mice. Blood. 117:2223-6.

6 Zambidis E T, Scott D W. 1996. Epitope-specific tolerance induction with an engineered immunoglobulin. Proc Natl Acad Sci USA. 93:5019-24.

7 Brumeanu T D, Casares S, Harris P E, Dehazya P, Wolf I, von Boehmer H, Bona C A. 1996. Immunopotency of a viral peptide assembled on the carbohydrate moieties of self immunoglobulins. Nat Biotechnol. 14:722-5.

8 De Groot A S, Moise L, McMurry J A, Wambre E, Van Overtvelt L, Moingeon P, Scott D W, Martin W. 2008. Activation of natural regulatory T cells by IgG Fc-derived peptide “Tregitopes”. Blood. 112:3303-11.

9 Legge K L, Gregg R K, Maldonado-Lopez R M, et al. 2002. On the role of dendritic cells in peripheral T cell tolerance and modulation of autoimmunity. J Exp Med. 196:217-227.

10 Melo M E, Qian J, El-Amine M, et al. 2002. Gene transfer of Ig-fusion proteins into B cells prevents and treats autoimmune diseases. J Immunol. 168:4788-95.

11 Lassila O, Vainio O, Matzinger P. 1988. Can B cells turn on virgin T cells? Nature. 334:253-5.

12 Eynon E E, Parker D C. 1992. Small B cells as antigen-presenting cells in the induction of tolerance to soluble protein antigens. J Exp Med. 175:131-8.

13 Fuchs E J, Matzinger P. 1992. B cells turn off virgin but not memory T cells. Science. 258:1156-9.

14 Sonoda K H, Stein-Streilein J. 2002. CD1d on antigen-transponting APC and splenic marginal zone B cells promotes NKT cell-dependent tolerance. Eur J immunol. 32:848-57.

15 Schultz J, Lin Y, Sanderson J, et al. 2000. A tetravalent single-chain antibody-streptavidin fusion protein for pretargeted lymphoma therapy. Cancer Res. 60:6663-9.

16 Borel Y, Lewis R M, Stollar B D. 1973. Prevention of murine lupus nephritis by carrier-dependent induction of immunologic tolerance to denatured DNA. Science. 182:76-8.

17 Borel Y. 1980. Haptens bound to self IgG induce immunologic tolerance, while when coupled to syngeneic spleen cells they induce immune suppression. Immunol Rev. 50:71-104.

18 Canfield S M, and Morrison S L. 1991. The binding affinity of human IgG for its high affinity Fc receptor is determined by multiple amino acids in the C_{H}2 domain and is modulated by the hinge region. J. Exp. Med. 173:1483-1491.

19 Gong Q, Ou Q, Ye S, et al. 2005. Importance of cellular microenvironment and circulatory dynamics in B cell immunotherapy. J Immunol. 174:817-26.

20 Offner H, Burrows G G, Ferro A J, et al. 2011. RTL therapy for multiple sclerosis: a phase I clinical study. J Neuroimmunol. 231:7-14.

21 Zhang A H, Li X, Onabajo O O. 2010. B-cell delivered gene therapy for tolerance induction: role of autoantigen-specific B cells. J Autoimmunity. 35:107-13.

22 Beers S A, French R R, Claude Chan H T, et al. 2010. Antigenic modulation limits the efficacy of anti-CD20 antibodies: implications for antibody selection. Blood. 115:5191-5201.

23 Xu B, Scott D W. 2004. A novel retroviral gene therapy approach to inhibit specific antibody production and suppress experimental autoimmune encephalomyelitis induced by MOG and MBP. Clin Immunol. 111:47-52.

24 Su Y, Zhang A H, Li X, et al. 2011. B cells “transduced” with TAT-fusion proteins can induce tolerance and protect mice from diabetes and EAE. Clin Immunol. 140:260-67.

25 Zhou L J, Smith H M, Waldschmidt T J, et al. 1994. Tissue-specific expression of the human CD19 gene in transgenic mice inhibits antigen-independent B-lymphocyte development. Mol Cell Biol. 14:3884-94.

26. X. Wang et al., Immune tolerance induction to factor IX through B cell gene transfer: TLR9 signaling delineates between tolerogenic and immunogenic B cells. Mol Ther 22, 1139-1150 (2014).

1.-28. (canceled)

29. An isolated nucleic acid molecule comprising a nucleotide sequence encoding a fusion protein, wherein said fusion protein comprises an antigen, an IgG heavy chain constant region or a fragment thereof, and a B cell surface targeting molecule.

30. The isolated nucleic acid molecule of claim 29, wherein said IgG heavy chain constant region is a modified human IgG4 heavy chain constant region.

31. The isolated nucleic acid molecule of claim 30 wherein said fusion protein does not exhibit B cell depleting efficacy.

32. The isolated nucleic acid molecule of claim 31, wherein said IgG4 heavy chain constant region lacks a hinge region or the CH1 region.

33. The isolated nucleic acid molecule of claim 32, wherein said B cell surface targeting molecule is an anti-CD20 single chain variable fragment or an anti-CD19 single chain variable fragment.

34. The isolated nucleic acid molecule of claim 33, wherein said B cell surface targeting molecule is a humanized anti-CD20 single chain variable fragment comprising an anti-CD20 variable heavy region linked to an anti-CD20 variable light region.

35. The isolated nucleic acid molecule of claim 34, wherein said heavy and light regions are linked via a linker comprising the amino acid sequence (Gly-Gly-Gly-Gly-Ser)₃ (SEQ ID NO: 1).

36. The isolated nucleic acid molecule of claim 35, wherein said protein antigen is selected from the group consisting of myelin basic protein (MBP), myelin oligodendrocyte glycoprotein (MOG), proteolipid protein (PLP), Factor VIII C2 domain, Factor VIII A2 domain, or fragments of FVIII domains.

37. An expression vector comprising the nucleic acid molecule of claim 36.

38. A host cell comprising the expression vector of claim 37.

39. A fusion protein comprising a protein antigen, an IgG heavy chain constant region or a fragment thereof, and a B cell surface targeting molecule.

40. The fusion protein of claim 39, wherein said IgG heavy chain constant region is a modified human IgG4 heavy chain constant region lacking a hinge region or the CH1 region.

41. The fusion protein of claim 39, wherein said B cell surface targeting molecule is an anti-CD20 single chain variable fragment or an anti-CD19 single chain variable fragment.

42. The fusion protein of claim 39, wherein said B cell surface targeting molecule is a humanized anti-CD20 single chain variable fragment comprising an anti-CD20 variable heavy region linked to an anti-CD20 variable light region.

43. The fusion protein of claim 39, wherein said protein antigen is selected from the group consisting of myelin basic protein (MBP), myelin oligodendrocyte glycoprotein (MOG), proteolipid protein (PLP), Factor VIII C2 domain, Factor VIII A2 domain, or fragments of FVIII domains.

44. A method of inducing tolerogenicity to an endogenous protein in an individual by administering the fusion protein of claim 39 to said individual.

45. A method of inducing tolerogenicity to an endogenous protein in an individual by administering the isolated nucleic acid molecule of claim 29 to said individual.

46. The method of claim 44, further comprising administering a B cell depletion agent.

47. The method of claim 46, wherein said B cell depletion agent reduces the amount of all types of B cells.

48. The method of claim 46, wherein said B cell depletion agent is rituximab or equivalent.

49. The method of claim 46, wherein said B cell depletion agent selectively reduces the amount of follicular B cells and does not reduce the amount of marginal zone B cells or reduces the amount of marginal zone B cells to a lesser extent that follicular B cells.

50. The method of claim 49, wherein said B cell depletion agent is a human equivalent mouse IgG1 isotype anti-CD20 monoclonal antibody.

51. The method of claim 44, wherein said endogenous protein is selected from the group consisting of MBP, MOG, PLP, Factor VIII C2 domain and Factor VIII A2 domain.

52. The method of claim 51, wherein said endogenous protein is MOG and said antigen comprises amino acid residues 35-55 of MOG.

53. The method of claim 44, wherein said individual has been diagnosed with multiple sclerosis.

54. The method of claim 44, wherein said individual has been diagnosed with one of the following diseases, uveitis, type 1 diabetes, arthritis, myasthenia gravis, hemophilia A or B and multiple sclerosis, but also could be used for monogenic enzyme deficiency diseases, such as Pompe's.

55. The isolated nucleic acid molecule of claim 29, wherein the encoded fusion protein comprises, from the N-terminus to the C-terminus, a B cell surface targeting molecule, an antigen, and an IgG heavy chain constant region or a fragment thereof.

56. The fusion protein of claim 39, comprising, from the N-terminus to the C-terminus, a B cell surface targeting molecule, an antigen, and an IgG heavy chain constant region or a fragment thereof.

57. A host cell comprising the isolated nucleic acid molecule of claim 36.


SEQUENCE LISTING (33 sequences)

SEQ ID NO: 1 - protein, 15 aa, Artificial Sequence
  source: /note=“Description of Artificial Sequence Synthetic peptide”
GGGGSGGGGSGGGGS

SEQ ID NO: 2 - DNA, 714 bp, Artificial Sequence
  source: /note=“Description of Artificial Sequence Synthetic polynucleotide”
gaagtccagctgcagcagtctggggctgagcttgtgagacctgggacctctgtgaagttatcttgcaaagtttctggcgataccattacattttactacatgcactttgtgaagcaaaggcctggacagggtctggaatggataggaaggattgatcctgaggatgaaagtactaaatattctgagaagttcaaaaacaaggcgacactcactgcagatacatcttccaacacagcctacctgaagctcagcagcctgacctctgaggacactgcaacctatttttgtatctacggaggatactactttgattactggggccaaggggtcatggtcacagtctcctcaggtggaggtggatcaggtggaggtggatctggtggaggtggatctgacatccagatgacacagtctccagcttccctgtctacatctctgggagaaactgtcaccatccaatgtcaagcaagtgaggacatttacagtggtttagcgtggtatcagcagaagccagggaaatctcctcagctcctgatctatggtgcaagtgacttacaagacggcgtcccatcacgattcagtggcagtggatctggcacacagtattctctcaagatcaccagcatgcaaactgaagatgaaggggtttatttctgtcaacagggtttaacgtatcctcggacgttcggtggcggcaccaagctggaattgaaa

SEQ ID NO: 3 - DNA, 714 bp, Artificial Sequence
  source: /note=“Description of Artificial Sequence Synthetic polynucleotide”
gaggtccagctgcagcagagcggagcagaactggtccgtcccggcacaagcgtgaaactgtcatgtaaagtgtcaggcgatactattacattctactatatgcactttgtcaaacagaggccaggacagggactggagtggatcggacggattgaccccgaggatgaatctactaagtacagtgaaaagttcaaaaacaaggccaccctgacagctgacacctccagcaatacagcttatctgaaactgtctagtctgactagcgaggacactgcaacctacttctgcatctatggcggatactattttgattactggggtcagggcgtgatggtcaccgtgtcatctggaggtggaggatcaggaggtggaggctccggaggtggaggaagcgacattcagatgacccagagtcccgccagcctgtctacaagtctgggcgagacagtgactatccagtgtcaggcatctgaagacatctacagtggcctggcctggtatcagcagaaacccggcaagtcccctcagctgctgatctacggcgctagcgacctgcaggatggagtcccttctagattttcaggctccgggagcggtacccagtattcactgaagattacttccatgcagaccgaggatgaaggggtgtacttctgccagcagggactgacatatcctaggacttttgggggtggcacaaaactggaactgaag

SEQ ID NO: 4 - protein, 238 aa, Artificial Sequence
  source: /note=“Description of Artificial Sequence Synthetic polypeptide”
EVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELK

SEQ ID NO: 5 - DNA, 750 bp, Artificial Sequence
  source: /note=“Description of Artificial Sequence Synthetic polynucleotide”
atggctcaggttcagctggtccagtcaggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggtctattactgtgcaagagcgcaattacgacctaactactggtacttcgatgtctggggcgcagggaccacggtcaccgtgagcaagatctctggtggcggtggctcgggcggtggtgggtcgggtggcggaggctcgggtggctcgagcgacatcgtgctgtcgcagtctccagcaatcctgtctgcatctccaggggagaaggtcacaatgacttgcagggccagctcaagtgtaagttacatgcactggtaccagcagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggattagtaacccacccacgttcggtgctgggaccaagctggagctgaag

SEQ ID NO: 6 - DNA, 729 bp, Artificial Sequence
  source: /note=“Description of Artificial Sequence Synthetic polynucleotide”
atggctcaggtgcagctggtgcagagtggggcagaactggtgaaacccggcgcaagcgtgaagatgtcatgtaaagcaagtggctacactttcaccagctacaacatgcactgggtgaaacagacaccaggacagggactggagtggatcggagctatttaccctgggaacggtgacacttcttataatcagaagtttaaaggcaaggctacactgactgctgataagtccagctctactgcttatatgcagctgagttcactgaccagtgaagactcagcagtgtactattgcgcaagggcccagctgcggcccaattactggtatttcgatgtctggggcgcaggaaccacagtgaccgtctccggaggaggaggtagtggaggaggaggtagcggcggagggggttctgacatcgtgctgtctcagagtcctgccattctgtcagcttccccaggcgagaaagtgaccatgacatgtcgagcctccagctctgtctcctacatgcattggtatcagcagaaacctggcagttcaccaaagccctggatctacgcaacctccaacctggccagcggagtgccagctagattcagcgggtctggtagtggcacttcatattccctgaccatctcccgcgtcgaggccgaagatgccgctacctactattgccagcagtggatttccaacccccctacatttggagccgggactaaactggaactgaag

SEQ ID NO: 7 - protein, 243 aa, Artificial Sequence
  source: /note=“Description of Artificial Sequence Synthetic polypeptide”
MAQVQLVQSGAELVKPGASVKMSCKASGYTFTSYNMHWVKQTPGQGLEWIGAIYPGNGDTSYNQKFKGKATLTADKSSSTAYMQLSSLTSEDSAVYYCARAQLRPNYWYFDVWGAGTTVTVSGGGGSGGGGSGGGGSDIVLSQSPAILSASPGEKVTMTCRASSSVSYMHWYQQKPGSSPKPWIYATSNLASGVPARFSGSGSGTSYSLTISRVEAEDAATYYCQQWISNPPTFGAGTKLELK

SEQ ID NO: 8 - DNA, 60 bp, Artificial Sequence
  source: /note=“Description of Artificial Sequence Synthetic oligonucleotide”
acagcttccagctacttcaccaatatgtttgcaacatggagcccctctaaggccaggctg

SEQ ID NO: 9 - protein, 20 aa, Artificial Sequence
  source: /note=“Description of Artificial Sequence Synthetic peptide”
TASSYFTNMFATWSPSKARL

SEQ ID NO: 10 - DNA, 51 bp, Artificial Sequence
  source: /note=“Description of Artificial Sequence Synthetic oligonucleotide”
atctcccaggcagtgcacgcagcacatgctgagattaatgaagcaggcagg

SEQ ID NO: 11 - protein, 17 aa, Artificial Sequence
  source: /note=“Description of Artificial Sequence Synthetic peptide”
ISQAVHAAHAEINEAGR

SEQ ID NO: 12 - DNA, 63 bp, Artificial Sequence
  source: /note=“Description of Artificial Sequence Synthetic oligonucleotide”
atggaagtcgggtggtatagaagccctttttcacgggtcgtccatctgtatcggaacggcaaa

SEQ ID NO: 13 - protein, 21 aa, Artificial Sequence
  source: /note=“Description of Artificial Sequence Synthetic peptide”
MEVGWYRSPFSRVVHLYRNGK

SEQ ID NO: 14 - DNA, 1104 bp, Artificial Sequence
  source: /note=“Description of Artificial Sequence Synthetic polynucleotide”
tccgtcgctaagaaacatcctaaaacctgggtgcattacatcgctgctgaagaagaagactgggactacgcccctctggtgctggcacccgacgatcgatcatacaaatcccagtatctgaacaatgggcctcagcgaatcggtcgtaagtacaagaaagtgaggttcatggcttatacagatgagacttttaaaacccgggaggcaatccagcacgaatccggcattctgggacctctgctgtacggggaagtgggtgacacactgctgatcatttttaagaaccaggcaagcagaccttacaatatctatccacacgggattactgatgtgcgcccactgtactctaggcggctgcccaagggggtcaaacatctgaaggacttcccaatcctgcccggcgagatttttaagtataaatggaccgtgacagtcgaagatggacccacaaagagtgaccctaggtgcctgactcggtactattccagcttcgtgaacatggagagagatctggcttccggcctgatcggaccactgctgatttgttacaaagaatcagtcgatcagagaggcaaccagatcatgtccgacaagcgcaatgtgattctgttctccgtctttgacgagaatcgaagctggtatctgacagaaaacatccagcgtttcctgcccaatcctgcaggagtgcagctggaggaccccgaatttcaggcttccaacatcatgcacagcattaatggatacgtcttcgacagcctgcagctgtctgtgtgcctgcatgaggtcgcatactggtatatcctgagcattggcgcccagaccgatttcctgagtgtgttcttttcaggatacaccttcaagcacaaaatggtctatgaggacactctgaccctgttccctttttctggcgagaccgtgtttatgagtatggaaaacccaggcctgtggattctgggatgccataactctgatttcagaaatcgcgggatgactgccctgctgaaagtgtctagttgtgacaagaataccggtgactactatgaggattcttacgaagacatcagtgcctatctgctgtcaaagaacaatgctattgagccaagg

SEQ ID NO: 15 - protein, 368 aa, Artificial Sequence
  source: /note=“Description of Artificial Sequence Synthetic polypeptide”
SVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPR

SEQ ID NO: 16 - DNA, 480 bp, Artificial Sequence
  source: /note=“Description of Artificial Sequence Synthetic polynucleotide”
agttgttcaatgccactgggtatggagtcaaaagcaatttccgacgcacagatcaccgcctcatcctacttcaccaatatgttcgctacctggagtccctcaaaggcccgactgcacctgcagggcagaagcaatgcttggcgcccacaggtgaacaatcccaaagagtggctgcaggtcgacttccagaagactatgaaagtgaccggcgtcaccacacagggagtgaagtccctgctgaccagcatgtacgtcaaagaatttctgatctccagctctcaggatggacatcagtggacactgttctttcagaacgggaaggtgaaagtcttccagggtaatcaggactcttttacacctgtggtcaacagtctggacccccctctgctgactaggtacctgagaatccacccacagtcctgggtgcatcagattgcactgaggatggaggtgctgggctgcgaagcccaggacctgtat

SEQ ID NO: 17 - protein, 160 aa, Artificial Sequence
  source: /note=“Description of Artificial Sequence Synthetic polypeptide”
SCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 18 - DNA, 1158 bp, Artificial Sequence
  source: /note=“Description of Artificial Sequence Synthetic polynucleotide”
atggggtcaatcggggccgccagtatggagttttgtttcgacgtctttaaggagctgaaggtccaccacgccaacgagaacattttttactgccccatcgcaattatgtctgccctggctatggtgtatctgggagccaaggacagtaccagaacacagatcaacaaggtggtccgcttcgacaaactgccagggtttggcgactccattgaggctcagtgtgggactagcgtgaatgtccactccagcctgcgggacatcctgaaccagattaccaagcccaatgatgtgtacagcttctctctggcatccaggctgtacgccgaggaacggtatcccatcctgcctgagtacctgcagtgcgtgaaagaactgtataggggcggactggagcctatcaactttcagacagccgctgaccaggcccgggaactgattaattcttgggtggagagtcagactaacggtatcattagaaatgtgctgcagccatctagtgtcgattcccagaccgctatggtgctggtcaacgctatcgtgttcaagggcctgtgggagaagaccttcaaggacgaagatactcaggctatgccattccgcgtgacagagcaggaatccaaacccgtccagatgatgtatcagatcggcctgttccgagtggctagcatggccagcgagaagatgaaaattctggaactgccttttgcctcaggaactatgtccatgctggtgctgctgccagacgaggtcagtgggctggagcagctggaatcaatcattaacttcgagaagctgactgaatggacctcatccaatgtgatggaggaacgaaagatcaaagtctacctgcctcgtatgaagatggaggaaaaatataacctgaccagcgtgctgatggctatgggtattacagatgtgtttagctctagtgcaaatctgtctggcatctcatccgccgagagcctgaagatttctcaggctgtgcacgcagcccatgctgagatcaacgaagcaggccgtgaggtggtcggaagcgcagaagcaggagtggacgctgcaagtgtctcagaggagttcagggccgatcaccccttcctgttttgcatcaaacatattgccacaaatgctgtgctgttctttggaagatgtgtctctcct

SEQ ID NO: 19 - protein, 386 aa, Artificial Sequence
  source: /note=“Description of Artificial Sequence Synthetic polypeptide”
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKTFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 20 - DNA, 25 bp, Artificial Sequence
  source: /note=“Description of Artificial Sequence Synthetic primer”
agaggaagcttatggctcaggttca

SEQ ID NO: 21 - DNA, 25 bp, Artificial Sequence
  source: /note=“Description of Artificial Sequence Synthetic primer”
agagcgaattccttcagctccagct

SEQ ID NO: 22 - DNA, 21 bp, Artificial Sequence
  source: /note=“Description of Artificial Sequence Synthetic primer”
agagagaattccttccaccaa

SEQ ID NO: 23 - DNA, 26 bp, Artificial Sequence
  source: /note=“Description of Artificial Sequence Synthetic primer”
acccacactagttttacccagagaca

SEQ ID NO: 24 - DNA, 36 bp, Artificial Sequence
  source: /note=“Description of Artificial Sequence Synthetic primer”
agtccaaatatggtcccccatgcccatcatgcccag

SEQ ID NO: 25 - protein, 21 aa, Artificial Sequence
  source: /note=“Description of Artificial Sequence Synthetic peptide”
MEVGWYRSPFSRVVHLYRNGK

SEQ ID NO: 26 - DNA, 23 bp, Artificial Sequence
  source: /note=“Description of Artificial Sequence Synthetic primer”
agcagactagtatggaggtgggt

SEQ ID NO: 27 - DNA, 27 bp, Artificial Sequence
  source: /note=“Description of Artificial Sequence Synthetic primer”
attatgcggccgccttgccatttcggt

SEQ ID NO: 28 - DNA, 26 bp, Artificial Sequence
  source: /note=“Description of Artificial Sequence Synthetic primer”
agcgcactagtaagatatctcaagct

SEQ ID NO: 29 - DNA, 27 bp, Artificial Sequence
  source: /note=“Description of Artificial Sequence Synthetic primer”
attatgcggccgcgcctgcttcattga

SEQ ID NO: 30 - DNA, 22 bp, Artificial Sequence
  source: /note=“Description of Artificial Sequence Synthetic primer”
actccactagtatggtgagcaa

SEQ ID NO: 31 - DNA, 27 bp, Artificial Sequence
  source: /note=“Description of Artificial Sequence Synthetic primer”
attatgcggccgccttgtacagctcgt

SEQ ID NO: 32 - protein, 6 aa, Artificial Sequence
  source: /note=“Description of Artificial Sequence Synthetic 6xHis tag”
HHHHHH

SEQ ID NO: 33 - protein, 5 aa, Artificial Sequence
  source: /note=“Description of Artificial Sequence Synthetic peptide”
GGGGS